(19) United States
(12) Patent Application Publication
(10) Pub. No.: US 2024/0254213 A1
(43) Pub. Date: Aug. 1, 2024

(54) GDF15 MODULATOR FOR USE IN INHIBITION OF OCULAR TISSUE FIBROSIS

(71) Applicants: Hideaki HARA, Gifu (JP); Masamitsu SHIMAZAWA, Gifu (JP); Shinsuke NAKAMURA, Gifu (JP)

(72) Inventors: Hideaki HARA, Gifu (JP); Masamitsu SHIMAZAWA, Gifu (JP); Shinsuke NAKAMURA, Gifu (JP)

(21) Appl. No.: 18/034,293

(22) PCT Filed: Oct. 29, 2021
(86) PCT No.: PCT/JP2021/040902

§ 371 Date: Dec. 12, 2023

(30) Foreign Application Priority Data
Oct. 30, 2020 (JP) 2020-182538

(51) Int. Cl. C07K 16/22; A61K 39/00; A61P 27/02

(52) U.S. Cl. CPC C07K 16/22; A61P 27/02; A61K 2039/505; C07K 2317/24; C07K 2317/565; C07K 2317/76

(57) ABSTRACT

The invention provides novel methods of treating ocular disorders that cause ocular tissue fibrosis by using a substance that inhibits the action of GDF15 (growth differentiation factor 15). In the present invention, the substance inhibiting the action of the GDF15 may be an anti-GDF15 antibody or an antagonist of a GDF15-specific receptor. Furthermore, in the present invention, the ocular tissue may be retinal pigment epithelial cells.

### CROSS-REFERENCE TO RELATED APPLICATIONS

This application is a 371 Application of International Application No. PCT/JP2021/040902, filed on Oct. 29, 2021, which claims priority to Japanese Patent Application No. 2020-182538 filed with the Japanese Patent Office on Oct. 30, 2020, which application is hereby incorporated by reference herein in its entirety.

### REFERENCE TO A SEQUENCE LISTING

The instant application contains a Sequence Listing in .txt. format which has been submitted via EFS-Web and is herein incorporated by reference in its entirety. The Sequence Listing, created on May 3, 2023 is named AVO-043US_SL_ST25.txt and is 206,682 bytes in size.

### FIELD OF THE INVENTION

The present invention relates to GDF15 modulators and their use in inhibiting ocular tissue fibrosis.

### BACKGROUND OF THE INVENTION

Age-related macular degeneration is currently one of the main causes of blindness in developed countries and is found mainly in elderly people aged 50 and over. Age-related macular degeneration is a disease caused by age-related changes in the macula and is broadly classified into exudative age-related macular degeneration, atrophic age-related macular degeneration, and early age-related macular degeneration, which is a precursor for these diseases. Exudative age-related macular degeneration is a disease in which neovascularization occurs in the macula starting at the choroid, bleeding or exudative lesions occur in the retinal pigment epithelium or beneath the retina, and finally fibrous scar tissue is formed. Fibrous scar tissue formed beneath the retina leads to irreversible visual field defects and blindness.

One method for treating exudative age-related macular degeneration is drug treatment. In drug treatment, a drug that inhibits vascular endothelial growth factor (VEGF) is injected into the vitreous body to suppress edema. Vascular endothelial cell growth factor is a factor believed to induce neovascularization. Examples of this drug include Lucentis®, Eylea®, Beovu®, and the like.

However, the drugs described above do not act on fibrous scar tissue formed beneath the retina. On the other hand, it has been reported that fibrous scarring of the macula occurs after drug treatment (Daniel E. et al., Ophthalmology, 2014 March, 121 (3), pp. 656-666). Furthermore, it is reported that formation of fibrous scar tissue is observed two years after beginning drug treatment in about half of patients (Daniel E. et al., Ophthalmology, 2018 July, 125 (7), pp. 1037-1046).

As described above, as conventional drugs used for exudative age-related macular degeneration do not act on fibrous scar tissue formed under the retina, there is currently no specific treatment for this fibrous scar tissue. The formation of fibrous scar tissue occurs not only in the exudative age-related macular degeneration described above, but also in intractable vitreoretinal diseases such as proliferative vitreoretinopathy and diabetic retinopathy, and even when retinal bleeding and retinal detachment are cured by treatment, subsequently secondarily formed fibrous scar tissue in the retinal choroid may lead to poor visual acuity. Therefore, how to suppress formation of fibrous scar tissue in the retinal choroid is important.

An object of the present invention is to provide a novel ocular tissue fibrosis inhibitor for such circumstances.

### SUMMARY OF THE INVENTION

In one aspect, the invention provides a method of reducing ocular tissue fibrosis in a subject in need thereof. The method comprises administering to the subject an effective amount of a GDF15 modulator thereby to reduce ocular tissue fibrosis in the subject. In one embodiment, the ocular tissue fibrosis is reduced in retinal pigment epithelial cells.

In another aspect, the invention provides a method of treating an ocular fibrosis disorder in a subject in need thereof. The method comprises administering to the subject an effective amount of a GDF15 modulator thereby to treat the disorder in the subject. In certain embodiments, the disorder is selected from macular degeneration (e.g., wet age-related macular degeneration), an intractable retinal vitreous disease (e.g., proliferative vitreoretinopathy or diabetic retinopathy), diabetic macular edema (DME), retinal hemorrhage, retinal detachment, presbyopia, choroidal neovascularization, subfoveal or juxtafoveal neovascularization, corneal astigmatism, and lenticular astigmatism.

In certain embodiments of the foregoing methods, the GDF15 modulator decreases or inhibits GDF15 activity. In certain embodiments, the GDF15 modulator is an anti-GDF15 antibody, for example, a humanized or human antibody. In certain embodiments, the anti-GDF15 antibody is selected from:
(i) an antibody comprising a CDR_{H1} sequence of SEQ ID NO:1, a CD_{H2} sequence of SEQ ID NO:7, and a CDR_{H3} sequence of SEQ ID NO:13; and a CDR_{L1} sequence of SEQ ID NO: 16, a CDR_{L2} sequence of SEQ ID NO: 18, and a CDR_{L3} sequence of SEQ ID NO:22;
(ii) an antibody comprising a CDR_{H1} sequence of SEQ ID NO:1, a CD_{H2} sequence of SEQ ID NO:9, and a CDR_{H3} sequence of SEQ ID NO: 13; and a CDR_{L1} sequence of SEQ ID NO: 16, a sequence of SEQ ID NO: 18, and a CDR_{L3} sequence of SEQ ID NO:22;
(iii) an antibody comprising a CDR_{H1} sequence of SEQ ID NO:1, a CD_{H2} sequence of SEQ ID NO:4, and a CDR_{H3} sequence of SEQ ID NO: 13; and a CDR_{L1} sequence of SEQ ID NO:16, a CDR_{L2} sequence of SEQ ID NO: 18, and a CDR_{L3} sequence of SEQ ID NO:21;
(iv) an antibody comprising a CDR_{H1} sequence of SEQ ID NO:1, a CD_{H2} sequence of SEQ ID NO:5, and a CDR_{H3} sequence of SEQ ID NO:13; and a CDR_{L1} sequence of SEQ ID NO:16, a CDR_{L2} sequence of SEQ ID NO:18, and a CDR_{L3} sequence of SEQ ID NO:21;
(v) an antibody comprising a CDR_{H1} sequence of SEQ ID NO: 1, a CD_{H2} sequence of SEQ ID NO:6, and a CDR_{H3} sequence of SEQ ID NO:13; and a CDR_{L1} sequence of SEQ ID NO: 16, a CDR_{L2} sequence of SEQ ID NO:18, and a CDR_{L3} sequence of SEQ ID NO:21;
(vi) an antibody comprising a CDR_{H1} sequence of SEQ ID NO: 1, a CD_{H2} sequence of SEQ ID NO:8, and a CDR_{H3} sequence of SEQ ID NO: 13; and a CDR_{L1} sequence of SEQ ID NO: 16, a CDR_{L2} sequence of SEQ ID NO: 18, and a CDR_{L3} sequence of SEQ ID NO:21;
(vii) an antibody comprising a CDR_{H1} sequence of SEQ ID NO:1, a CD_{H2} sequence of SEQ ID NO:9, and a CDR_{H3} sequence of SEQ ID NO: 13; and a CDR_{L1} sequence of SEQ ID NO:16, a CDR_{L2} sequence of SEQ ID NO: 18, and a CDR_{L3} sequence of SEQ ID NO:21;
(viii) an antibody comprising the heavy chain sequence of SEQ ID NO:47 or 49, and the light chain sequence of SEQ ID NO:30;
(ix) an antibody comprising the heavy chain sequence of SEQ ID NO:41, 42, 43, 44, 45, 46, 48, or 49 or a variable region thereof, and the light chain sequence of SEQ ID NO:29 or the variable region thereof;
(x) an antibody comprising the heavy chain sequence of SEQ ID NO:41, 42, 43, 44, or 45, and the light chain sequence of SEQ ID NO:28 or the variable region thereof;
(xi) an antibody comprising the heavy chain sequence of SEQ ID NO:39, 40, 41, 42, 43, 44, or 45, or a variable region thereof, and the light chain sequence of SEQ ID NO:27 or the variable region thereof;
(xii) an antibody comprising the heavy chain sequence of SEQ ID NO:38 or the variable region thereof, and the light chain sequence of SEQ ID NO:26 or the variable region thereof;
(xiii) an antibody comprising the heavy chain sequence of SEQ ID NO:37or the variable region thereof, and the light chain sequence of SEQ ID NO:25 or the variable region thereof;
(xiv) an antibody comprising the heavy chain sequence of SEQ ID NO: 48 or the variable region thereof, and the light chain sequence of SEQ ID NO: 29 or the variable region thereof,; and
(xv) an antibody comprising the heavy chain sequence of SEQ ID NO: 47 or the variable region thereof, and the light chain sequence of SEQ ID NO: 30 or the variable region thereof.

In another aspect, the invention provides a GDF15 modulator, for use in reducing ocular tissue fibrosis in a subject in need thereof. In another aspect, the invention provides a GDF15 modulator, for use in the manufacture of a medicament for reducing ocular tissue fibrosis in a subject in need thereof.

In another aspect, the invention provides a GDF15 modulator, for use in the manufacture of a medicament for treating an ocular fibrosis disorder in a subject in need thereof.

In another aspect, the invention provides a GDF15 modulator, for use in treating an ocular fibrosis disorder in a subject in need thereof. In certain embodiments, the disorder is selected from macular degeneration (e.g., wet age-related macular degeneration), an intractable retinal vitreous disease (e.g., proliferative vitreoretinopathy or diabetic retinopathy), diabetic macular edema (DME), retinal hemorrhage, retinal detachment, presbyopia, choroidal neovascularization, subfoveal or juxtafoveal neovascularization, corneal astigmatism, and lenticular astigmatism.

In certain embodiments, the GDF15 modulator decreases or inhibits GDF15 activity. In certain embodiments, the GDF15 modulator is an anti-GDF15 antibody, for example, a humanized or human antibody. In certain embodiments, the anti-GDF15 antibody is selected from:
(i) an antibody comprising a CDR_{H1} sequence of SEQ ID NO:1, a CD_{H2} sequence of SEQ ID NO:7, and a CDR_{H3} sequence of SEQ ID NO:13; and a CDR_{L1} sequence of SEQ ID NO: 16, a CDR_{L2} sequence of SEQ ID NO: 18, and a CDR_{L3} sequence of SEQ ID NO:22;
(ii) an antibody comprising a CDR_{H1} sequence of SEQ ID NO:1, a CD_{H2} sequence of SEQ ID NO:9, and a CDR_{H3} sequence of SEQ ID NO: 13; and a CDR_{L1} sequence of SEQ ID NO: 16, a sequence of SEQ ID NO: 18, and a CDR_{L3} sequence of SEQ ID NO:22;
(iii) an antibody comprising a CDR_{H1} sequence of SEQ ID NO: 1, a CD_{H2} sequence of SEQ ID NO:4, and a CDR_{H3} sequence of SEQ ID NO: 13; and a CDR_{L1} sequence of SEQ ID NO: 16, a CDR_{L2} sequence of SEQ ID NO:18, and a CDR_{L3} sequence of SEQ ID NO:21;
(iv) an antibody comprising a CDR_{H1} sequence of SEQ ID NO:1, a CD_{H2} sequence of SEQ ID NO:5, and a CDR_{H3} sequence of SEQ ID NO: 13; and a CDR_{L1} sequence of SEQ ID NO: 16, a CDR_{L2} sequence of SEQ ID NO:18, and a CDR_{L3} sequence of SEQ ID NO:21;
(v) an antibody comprising a CDR_{H1} sequence of SEQ ID NO:1, a CD_{H2} sequence of SEQ ID NO:6, and a CDR_{H3} sequence of SEQ ID NO: 13; and a CDR_{L1} sequence of SEQ ID NO:16, a CDR_{L2} sequence of SEQ ID NO: 18, and a CDR_{L3} sequence of SEQ ID NO:21;
(vi) an antibody comprising a CDR_{H1} sequence of SEQ ID NO: 1, a CD_{H2} sequence of SEQ ID NO:8, and a CDR_{H3} sequence of SEQ ID NO:13; and a CDR_{L1} sequence of SEQ ID NO: 16, a CDR_{L2} sequence of SEQ ID NO: 18, and a CDR_{L3} sequence of SEQ ID NO:21;
(vii) an antibody comprising a CDR_{H1} sequence of SEQ ID NO: 1, a CD_{H2} sequence of SEQ ID NO:9, and a CDR_{H3} sequence of SEQ ID NO:13; and a CDR_{L1} sequence of SEQ ID NO:16, a CDR_{L2} sequence of SEQ ID NO: 18, and a CDR_{L3} sequence of SEQ ID NO:21;
(viii) an antibody comprising the heavy chain sequence of SEQ ID NO:47 or 49, and the light chain sequence of SEQ ID NO:30;
(ix) an antibody comprising the heavy chain sequence of SEQ ID NO:41, 42, 43, 44, 45, 46, 48, or 49, or a variable region thereof, and the light chain sequence of SEQ ID NO:29 or the variable region thereof;
(x) an antibody comprising the heavy chain sequence of SEQ ID NO:41, 42, 43, 44, or 45, or a variable region thereof, and the light chain sequence of SEQ ID NO:28 or the variable region thereof;
(xi) an antibody comprising the heavy chain sequence of SEQ ID NO:39, 40, 41, 42, 43, 44, or 45, or a variable region thereof, and the light chain sequence of SEQ ID NO:27 or the variable region thereof;
(xii) an antibody comprising the heavy chain sequence of SEQ ID NO:38 or the variable region thereof, and the light chain sequence of SEQ ID NO:26 or the variable region thereof;
(xiii) an antibody comprising the heavy chain sequence of SEQ ID NO:37 or the variable region thereof, and the light chain sequence of SEQ ID NO:25 or the variable region thereof;
(xiv) an antibody comprising the heavy chain sequence of SEQ ID NO: 48 or the variable region thereof, and the light chain sequence of SEQ ID NO: 29 or the variable region thereof; and
(xv) an antibody comprising the heavy chain sequence of SEQ ID NO: 47 or the variable region thereof, and the light chain sequence of SEQ ID NO: 30, or the variable region thereof.

In another aspect, the invention provides an ocular tissue fibrosis inhibitor containing a substance inhibiting the action of GDF15 (growth differentiation factor 15) as an active ingredient.

In certain embodiments, the substance inhibiting the action of the GDF15 may be an anti-GDF15 antibody or an antagonist of a GDF 15-specific receptor.

In certain embodiments, the ocular tissue may be retinal pigment epithelial cells.

### BRIEF DESCRIPTION OF DRAWINGS

The patent or application file contains at least one drawing executed in color. Copies of this patent or patent application with color drawing(s) will be provided by the Office upon request and payment of the necessary fee.

FIGS. 1A-D illustrate the results of example 1.

FIGS. 2A-D illustrate the results of example 2.

FIGS. 3A-B illustrate the results of example 3.

FIGS. 4A-F illustrate the results of example 4.

FIGS. 5A-C illustrate the results of example 5.

FIGS. 6A-C illustrate the results of example 6.

### DETAILED DESCRIPTION OF THE INVENTION

According to the present invention, novel methods of inhibiting ocular tissue fibrosis are provided.

Note, the effect described herein is not necessarily limited, and it may be any effect described in the present Specification.

Favorable embodiments of the present art will be described below.

The embodiments described below show one example of representative embodiments of the present art, and the scope of the present art is not to be narrowly interpreted due to such.

The ocular tissue fibrosis inhibitor of the present invention is characterized by containing a substance inhibiting the action of GDF15 (growth differentiation factor 15) as an active ingredient.

GDF15 is a secretory protein belonging to the TGFβ (transforming growth factor-β) superfamily and is known to have inhibitory actions on macrophages (Bottner, et al., Cell and Tissue Research. 1999, volume 297, pages 103-110). TGFβ is one type of cytokine known to suppress cell proliferation and differentiation and promote cell death. Heretofore, it has been reported that GDF15 induces epithelial mesenchymal transition of epithelial cancer cells via TGFβ receptors (Chen Li, et al., Oncotarget. 2016 Jan. 5; 7 (1):860-72) and is cleaved by Golgi body furin protease to become an activated form (Jing Jing Li, et al., Mol Cell Biol. 2018 Oct. 15; 38 (21):e00249-18. doi: 10.1128/MCB.00249-18. Print 2018 Nov. 1). Furthermore, in recent years, it has been reported that GFRAL (glial cell-derived neural factor family receptor alpha-like) receptors have been identified as high-affinity receptors for GDF15 (S. Mullican., et al., Nature Medicine, 23, pp. 1150-1157, 2017).

As described above, it is known that fibrous scar tissue in the retinal choroid, which is secondarily formed after treatment, causes poor visual acuity in intractable retinal vitreous diseases such as exudative age-related macular degeneration, proliferative vitreoretinopathy, and diabetic retinopathy, but there is currently no specific treatment for this fibrous scar tissue. Characteristics of fibrous scarring diseases include connective tissue and dense collagenous fibers having accumulated in excess, and in recent years, it is believed that epithelial mesenchymal transition (hereinafter also referred to as “EMT”) of lesion-peripheral cells contributes to fibrous scarring formation.

Epithelial mesenchymal transition is a process in which epithelial cells change into mesenchyme-like cells. This process transforms epithelial cells into myofibroblast-like cells, leading to fibrous scarring formation. It has been known that the expression of EMT-related factors is regulated by growth factors and cytokines, and thus the present inventors believe that the elucidation of EMT-related factors will lead to the elucidation of the pathology.

Also, as a result of diligent experiments and examination focusing on the GDF15 described above, as shown in the examples described below, the present inventors found that, because expression of GDF15 increased in fibrous scarring pathology, GDF15 is involved in the progression of fibrosis pathology by inducing epithelial mesenchymal transition. Accordingly, it became clear that a substance inhibiting the action of GDF15 can suppress fibrous scarring formation in the eye.

It is suggested that present invention is useful for radical treatment of ocular tissue fibrosis, and it is believed to contribute to the suppression of loss of visual acuity in patients for whom fibrous scarring formation is predicted and lead to rehabilitation of patients and reduction of medical expenses.

### GDF15 Modulators

In the present invention, “GDF15 modulator” or “substance inhibiting the action of GDF15” means a substance that reduces and/or inhibits the activity of GDF15 and/or the activity of the biological pathway of GDF15, which can result from a reduction of a level of expression, biological activity, biological function, and the like for GDF15 and/or in the biological pathway of GDF15. The substance inhibiting the action of the GDF15 is not particularly limited, and examples include an antagonist of GDF 15, an anti-GDF15 antibody, an antagonist of a GDF15-specific receptor, an anti-GDF15-specific receptor antibody, an inhibitor of a downstream signal of a GDF15-specific receptor, an expression inhibitor of GDF15, an expression inhibitor of a GDF15-specific receptor, a soluble GDF15 mimetic or analog that prevents GDF15 from binding to its cognate binding partner, a soluble GDF15 receptor mimetic or analog that prevents GDF15 from binding to its cognate binding partner, a small molecule inhibitor of GDF15 or of a GDF15 receptor, interfering nucleic acids (for example, interfering RNA or antisense nucleic acids (for example, antisense DNA or RNA) that interfere with expression of endogenous GDF15 or a cognate receptor, and the like. In the present invention, one type of these may be used, or two or more types may be used in combination. Furthermore, these substances may be obtained by refining known methods or may be acquired as commercially available products.

In a preferred embodiment, the GDF15 modulating agent can comprise an anti-GDF15 antibody or an anti-GDF15 receptor antibody, which is humanized or human. As used herein, unless otherwise indicated, the term “antibody” is understood to mean an intact antibody (e.g., an intact monoclonal antibody) or antigen-binding fragment of an antibody, including an intact antibody or antigen-binding fragment of an antibody (e.g., a phage display antibody including a fully human antibody, a semisynthetic antibody or a fully synthetic antibody) that has been optimized, engineered or chemically conjugated. Examples of antibodies that have been optimized are affinity-matured antibodies. Examples of antibodies that have been engineered are Fc optimized antibodies, and multispecific antibodies (e.g., bispecific antibodies). Examples of antigen-binding fragments include Fab, Fab′, F(ab′)2, Fv, single chain antibodies (e.g., scFv), minibodies and diabodies. An antibody conjugated to a toxin moiety is an example of a chemically conjugated antibody. Additional examples of antibodies include a polyclonal antibody, a polyspecific antibody, a bispecific antibody, a minibody, a domain antibody, a synthetic antibody, a chimeric antibody, an antibody fusion protein (also called an “antibody conjugate”) such as a fusion protein containing an antigen determining part of an antibody, fragments of each of these, and the like, but it is not limited to these.

In certain embodiments, the antibody comprises: (a) an immunoglobulin heavy chain variable region comprising the structure CDR_{H1}-CDR_{H2}-CDR_{H3} and (b) an immunoglobulin light chain variable region, wherein the heavy chain variable region and the light chain variable region together define a single binding site for binding GDF15 or a GDF15 receptor. The CDR_{H1}, CDR_{H2}, and CDR_{H3} sequences are interposed between immunoglobulin framework (FR) sequences. In certain other embodiments, the antibody comprises (a) an immunoglobulin light chain variable region comprising the structure CDR_{L1}-CDR_{L2}-CDR_{L3}, and (b) an immunoglobulin heavy chain variable region, wherein the IgG light chain variable region and the IgG heavy chain variable region together define a single binding site for binding GDF15 or a GDF15 receptor. The CDR_{L1}, CDR_{L2}, and CDR_{L3} sequences are interposed between immunoglobulin FR sequences. In certain other embodiments, the antibody comprises: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding GDF15 or a GDF15 receptor.

Exemplary anti-GDF15 antibodies useful in the methods and compositions of the invention may, for example, include a heavy chain variable region comprising any one of the nine sets of CDR_{H1}, CDR_{H2}, and CDR_{H3} region sequences set forth in Table 1 below.

TABLE 1

| | | | | Heavy Chain |
|---|---|---|---|---|
| | | | | Sequences |
| | | | | Containing |
| | | | | these CDRs |
| | CDR_{H1} | CDR_{H2} | CDR_{H3} | (SEQ ID NOS:) |
| 1 | DYNMD (SEQ ID | QINPNNGGIFFNQKFKG | EAITTVGAMDY (SEQ | 37, 38, 39, 40, |
| | NO: 1) | (SEQ ID NO: 4) | ID NO: 13) | 41, 44 |
| 2 | DYNMD (SEQ ID | QINPNNGGIFFNQKFQG | EAITTVGAMDY (SEQ | 42, 43, 45 |
| | NO: 1) | (SEQ ID NO: 5) | ID NO: 13) | |
| 3 | DYNMD (SEQ ID | QINPYNHLIFFNQKFQG | EAITTVGAMDY (SEQ | 46 |
| | NO: 1) | (SEQ ID NO: 6) | ID NO: 13) | |
| 4 | DYNMD (SEQ ID | QINPNNGLIFFNQKFQG | EAITTVGAMDY (SEQ | 47 |
| | NO: 1) | (SEQ ID NO: 7) | ID NO: 13) | |
| 5 | DYNMD (SEQ ID | QINPNNGLIFFNQKFKG | EAITTVGAMDY (SEQ | 48 |
| | NO: 1) | (SEQ ID NO: 8) | ID NO: 13) | |
| 6 | DYNMD (SEQ ID | QINPYNHLIFFNQKFKG | EAITTVGAMDY (SEQ | 49 |
| | NO: 1) | (SEQ ID NO: 9) | ID NO: 13) | |
| 7 | TYGMGVS (SEQ ID | HIYWDDDKRYNPSLKS | RGYDDYWGY (SEQ | |
| | NO: 2) | (SEQ ID NO: 10 | ID NO: 14) | |
| 8 | TYGMGVS (SEQ ID | HIYWDDDKRYNPSLKT | RGYDDYWGY (SEQ | |
| | NO: 2) | (SEQ ID NO: 11) | ID NO: 14) | |
| 9 | TYGMGVG (SEQ ID | DIW-WDDDKYYNPSLKS | RGHYSAMDY (SEQ | |
| | NO: 3) | (SEQ ID NO: 12) | ID NO: 15) | |

Exemplary anti-GDF15 antibodies useful in the methods and compositions of the invention may, for example, include a light chain variable region comprising any one of the four sets of CDR_{L1}, CDR_{L2}, and CDR_{L3} region sequences set forth in Table 2 below.

TABLE 2

| | | | | Light Chain Sequences |
|---|---|---|---|---|
| | | | | Containing These CDRs |
| | CDRL₁ | CDRL₂ | CDRL₃ | (SEQ ID NOS:) |
| 1 | RTSENLHNYLA | DAKTLAD (SEQ ID | QHFWSSPYT (SEQ ID | 25, 26, 27, 28, |
| | (SEQ ID NO: 16) | NO: 18) | NO: 21) | 29 |
| 2 | RTSENLHNYLA | DAKTLAD (SEQ ID | QHFWSDPYT (SEQ ID | 30 |
| | (SEQ ID NO: 16) | NO: 18) | NO: 22) | |
| 3 | KASQNVGTNVA | SASYRYS (SEQ ID | QQYNNYPLT (SEQ ID | |
| | (SEQ ID NO: 17) | NO: 19) | NO: 23) | |
| 4 | KASQNVGTNVA | SPSYRYS (SEQ ID | QQYNSYPHT (SEQ ID | |
| | (SEQ ID NO: 17) | NO: 20) | NO: 24) | |

Exemplary anti-GDF15 antibodies useful in the methods and compositions of the invention may, for example, include (i) any one of the nine sets of CDR_{H1}, CDR_{H2}, and CDR_{H3} region sequences set forth in Table 1, and (ii) any one of the four sets of CDR_{L1}, CDR_{L2}, and CDR_{L3} region sequences set forth in Table 2. For example, an exemplary anti-GDF15 antibody may comprise:
(i) a CDR_{H1} comprising the amino acid sequence of SEQ ID NO: 1, a CDR_{H2} comprising the amino acid sequence of SEQ ID NO: 7, a CDR_{H3}, comprising the amino acid sequence of SEQ ID NO: 13, a CDR_{L1} comprising the amino acid sequence of SEQ ID NO: 16, a CDR_{L2} comprising the amino acid sequence of SEQ ID NO: 18, and a CDR_{L3} comprising the amino acid sequence of SEQ ID NO: 22;
(ii) a CDR_{H1} comprising the amino acid sequence of SEQ ID NO: 1, a CDR_{H2} comprising the amino acid sequence of SEQ ID NO: 9, a CDR_{H3}, comprising the amino acid sequence of SEQ ID NO: 13, a CDR_{L1} comprising the amino acid sequence of SEQ ID NO: 16, a CDR_{L2} comprising the amino acid sequence of SEQ ID NO: 18, and a CDR_{L3} comprising the amino acid sequence of SEQ ID NO: 22;
(iii) a CDR_{H1} comprising the amino acid sequence of SEQ ID NO: 1, a CDR_{H2} comprising the amino acid sequence of SEQ ID NO: 4, a CDR_{H3}, comprising the amino acid sequence of SEQ ID NO: 13, a CDR_{L1} comprising the amino acid sequence of SEQ ID NO: 16, a CDR_{L2} comprising the amino acid sequence of SEQ ID NO: 18, and a CDR_{L3} comprising the amino acid sequence of SEQ ID NO: 21;
(iv) a CDR_{H1} comprising the amino acid sequence of SEQ ID NO: 1, a CDR_{H2} comprising the amino acid sequence of SEQ ID NO: 5, a CDR_{H3}, comprising the amino acid sequence of SEQ ID NO: 13, a CDR_{L1} comprising the amino acid sequence of SEQ ID NO: 16, a CDR_{L2} comprising the amino acid sequence of SEQ ID NO: 18, and a CDR_{L3} comprising the amino acid sequence of SEQ ID NO: 21;
(v) a CDR_{H1} comprising the amino acid sequence of SEQ ID NO: 1, a CDR_{H2} comprising the amino acid sequence of SEQ ID NO: 6, a CDR_{H3}, comprising the amino acid sequence of SEQ ID NO: 13, a CDR_{L1} comprising the amino acid sequence of SEQ ID NO: 16, a CDR_{L2} comprising the amino acid sequence of SEQ ID NO: 18, and a CDR_{L3} comprising the amino acid sequence of SEQ ID NO: 21;
(vi) a CDR_{H1} comprising the amino acid sequence of SEQ ID NO: 1, a CDR_{H2} comprising the amino acid sequence of SEQ ID NO: 8, a CDR_{H3}, comprising the amino acid sequence of SEQ ID NO: 13, a CDR_{L1} comprising the amino acid sequence of SEQ ID NO: 16, a CDR_{L2} comprising the amino acid sequence of SEQ ID NO: 18, and a CDR_{L3} comprising the amino acid sequence of SEQ ID NO: 21; or
(vii) a CDR_{H1} comprising the amino acid sequence of SEQ ID NO: 1, a CDR_{H2} comprising the amino acid sequence of SEQ ID NO: 9, a CDR_{H3}, comprising the amino acid sequence of SEQ ID NO: 13, a CDR_{L1} comprising the amino acid sequence of SEQ ID NO: 16, a CDR_{L2} comprising the amino acid sequence of SEQ ID NO: 18, and a CDR_{L3} comprising the amino acid sequence of SEQ ID NO: 21.

In one embodiment, the anti-GDF15 antibody comprises a CDR_{H1} comprising the amino acid sequence of SEQ ID NO: 1, a CDR_{H2} comprising the amino acid sequence of SEQ ID NO: 8, a CDR_{H3}, comprising the amino acid sequence of SEQ ID NO: 13, a CDR_{L1} comprising the amino acid sequence of SEQ ID NO: 16, a CDR_{L2} comprising the amino acid sequence of SEQ ID NO: 18, and a CDR_{L3} comprising the amino acid sequence of SEQ ID NO: 21.

In one embodiment, the anti-GDF15 antibody comprises a CDR_{H1} comprising the amino acid sequence of SEQ ID NO: 1, a CDR_{H2} comprising the amino acid sequence of SEQ ID NO: 7, a CDR_{H3}, comprising the amino acid sequence of SEQ ID NO: 13, a CDR_{L1} comprising the amino acid sequence of SEQ ID NO: 16, a CDR_{L2} comprising the amino acid sequence of SEQ ID NO: 18, and a CDR_{L3} comprising the amino acid sequence of SEQ ID NO: 22.

Exemplary anti-GDF-15 antibodies useful in the practice of the invention are described in U.S. Patent Application Publication No. 2014/0193427 (the disclosure of which is incorporated by reference herein for all purposes) including 01G06, 03G05, 04F08, 06C11, 08G01, 14F11, 17B11, as well as human or humanized forms thereof.

In a preferred embodiment, an anti-GDF-15 antibody useful in the practice of the invention is referred to as 01G06 in U.S. Patent Application Publication No. 2014/0193427. Humanized forms of the 01G06 antibody are listed below together with the amino acid sequences of their respective heavy and light chain regions. Exemplary humanized anti-GDF-15 antibodies include: Hu01G06-1; Hu01G06-46; Hu01G06-52; Hu01G06-100; Hu01G06-101; Hu01G06-102; Hu01G06-103; Hu01G06-104; Hu01G06-105; Hu01G06-106; Hu01G06-107; Hu01G06-108; Hu01G06-109; Hu01G06-110; Hu01G06-111; Hu01G06-112; Hu01G06-113; Hu01G06-114; Hu01G06-122; Hu01G06-127; Hu01G06-135; Hu01G06-138; Hu01G06-146; Hu06C11-1; Hu06C11-27; Hu06C11-30; Hu14F11-1; Hu14F11-23; Hu14F11-24; Hu14F11-39; and Hu14F11-47, and any antibody comprising the heavy and light chain variable regions of the foregoing antibodies. The amino acid sequences for the heavy chain and light chain for each of the aforementioned antibodies is set forth below in Table 3.

TABLE 3

| Antibody Name | Light Chain | Heavy Chain |
|---|---|---|
| 01G06 (murine) | SEQ ID NO:25 | SEQ ID NO:37 |
| Hu01G06-1 | SEQ ID NO:26 | SEQ ID NO:38 |
| Hu01G06-46 | SEQ ID NO:27 | SEQ ID NO:39 |
| Hu01G06-52 | SEQ ID NO:27 | SEQ ID NO:40 |
| Hu01G06-100 | SEQ ID NO:27 | SEQ ID NO:41 |
| Hu01G06-101 | SEQ ID NO:27 | SEQ ID NO:42 |
| Hu01G06-102 | SEQ ID NO:27 | SEQ ID NO:43 |
| Hu01G06-103 | SEQ ID NO:27 | SEQ ID NO:44 |
| Hu01G06-104 | SEQ ID NO:27 | SEQ ID NO:45 |
| Hu01G06-105 | SEQ ID NO:28 | SEQ ID NO:41 |
| Hu01G06-106 | SEQ ID NO:28 | SEQ ID NO:42 |
| Hu01G06-107 | SEQ ID NO:28 | SEQ ID NO:43 |
| Hu01G06-108 | SEQ ID NO:28 | SEQ ID NO:44 |
| Hu01G06-109 | SEQ ID NO:28 | SEQ ID NO:45 |
| Hu01G06-110 | SEQ ID NO:29 | SEQ ID NO:41 |
| Hu01G06-111 | SEQ ID NO:29 | SEQ ID NO:42 |
| Hu01G06-112 | SEQ ID NO:29 | SEQ ID NO:43 |
| Hu01G06-113 | SEQ ID NO:29 | SEQ ID NO:44 |
| Hu01G06-114 | SEQ ID NO:29 | SEQ ID NO:45 |
| Hu01G06-122 | SEQ ID NO:29 | SEQ ID NO:46 |
| Hu01G06-127 | SEQ ID NO:30 | SEQ ID NO:47 |
| Hu01G06-135 | SEQ ID NO:29 | SEQ ID NO:48 |
| Hu01G06-138 | SEQ ID NO:29 | SEQ ID NO:49 |
| Hu01G06-146 | SEQ ID NO:30 | SEQ ID NO:49 |
| 06C11 (murine) | SEQ ID NO:31 | SEQ ID NO:50 |
| Hu06C11-1 | SEQ ID NO:32 | SEQ ID NO:38 |
| Hu06C11-27 | SEQ ID NO:33 | SEQ ID NO:51 |
| Hu06C11-30 | SEQ ID NO:33 | SEQ ID NO:52 |
| 14F11 (murine) | SEQ ID NO:34 | SEQ ID NO:53 |
| Hu14F11-1 | SEQ ID NO:35 | SEQ ID NO:54 |
| Hu14F11-23 | SEQ ID NO:35 | SEQ ID NO:55 |
| Hu14F11-24 | SEQ ID NO:32 | SEQ ID NO:54 |
| Hu14F11-39 | SEQ ID NO:36 | SEQ ID NO:56 |
| Hu14F11-47 | SEQ ID NO:36 | SEQ ID NO:57 |

The following sequences are identified in Table 3 by their SEQ ID NOs. The variable regions of these heavy and light chain sequences are underlined and highlighted in boldface type.

| SEQ ID NO: 25 | |
|---|---|
| 1 | diqmtqspas lsasvgetvt itcrtsenlh nylawyqqkq gkspqllvyd aktladgvps |
| 61 | rfsgsgsgtq yslkinslqp edfgsyycgh fwsspytfgg gtkleikrad aaptvsifpp |
| 121 | sseqltsgga svvcflnnfy pkdinvkwki dgserqngvl nswtdqdskd stysmsstlt |
| 181 | ltkdeyerhn sytceathkt stspivksfn rnec |
| SEQ ID NO: 26 | |
| 1 | diqmtqspas lsasvgetvt itcrtsenlh nylawyqqkq gkspqllvyd aktladgvps |
| 61 | rfsgsgsgtq yslkinslqp edfgsyycgh fwsspytfgg gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |
| SEQ ID NO: 27 | |
| 1 | diqmtqspss lsasvgdrvt itcrtsenlh nylawyqqkp gkspkllvyd aktladgvps |
| 61 | rfsgsgsgtd ytltisslqp edfatyycqh fwsspytfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |
| SEQ ID NO: 29 | |
| 1 | diqmtqspss lsasvgdrvt itcrtsenlh nylawyqqkp gkapklliyd aktladgvps |
| 61 | rfsgsgsgtd ytltisslqp edfatyycqh fwsspytfgg gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |
| SEQ ID NO: 28 | |
| 1 | diqmtqspss lsasvgdrvt itcrtsenlh nylawyqqkp gkspklliyd aktladgvps |
| 61 | rfsgsgsgtd ytltisslqp edfatyycqh fwsspytfgg gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |
| SEQ ID NO: 32 | |
| 1 | divmtqsqkf mstsvgdrvs vtckasqnvg tnvawfqqkp gqspkaliys asyrysgvpd |
| 61 | rftgsgsgtd filtisnvqs edlaeyfcqg ynnypltfga gtklelkrtv aapsvfifpp |
| 121 | sdeqlksgta svvclInnfy preakvqwkv dnalqsgnsq esvteqdskd styslsstlt |
| 181 | lskadyekhk vyacevthqg lsspvtksfn rgec |
| SEQ ID NO: 33 | |
| 1 | diqmtqspss lsasvgdrvt itckasqnvg tnvawfqqkp gkapksliys asyrysgvps |
| 61 | rfsgsgsgtd ftltisslqp edfatyycqq ynnypltfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |
| SEQ ID NO: 35 | |
| 1 | divmtqsqkf mstsvgdrvs vtckasqnvg tnvawyqqkp gqspkaliys psyrysgvpd |
| 61 | rftgsgsgtd ftltisnvqs edlaeyfcqq ynsyphtfgg gtklemkrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |
| SEQ ID NO: 36 | |
| 1 | diqmtqspss lsasvgdrvt itckasqnvg tnvawfqqkp gkspkaliys psyrysgvps |
| 61 | rfsgsgsgtd ftltisslqp edfatyfcqq ynsyphtfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |
| SEQ ID NO: 37 | |
| 1 | evllqqsgpe lvkpgasvki pckasgytft dynmdwvkqs hgkslewigg inpnnggiff |
| 61 | nqkfkgkatl tvdkssntaf mevrsltsed tavyycarea ittvgamdyw gqgtsvtvss |
| 121 | akttppsvyp lapgsaaqtn smvtlgclvk gyfpepvtvt wnsgslssgv htfpavlqsd |
| 181 | lytlsssvtv psstwpsetv tcnvahpass tkvdkkivpr dcgckpcict vpevssvfif |
| 241 | ppkpkdvlti tltpkvtcvv vdiskddpev qfswfvddve vhtaqtqpre eqfnstfrsv |
| 301 | selpimhqdw lngkefkcrv nsaafpapie ktisktkgrp kapqvytipp pkeqmakdkv |
| 361 | sltcmitdff peditvewqw ngqpaenykn tqpimdtdgs yfvysklnvq ksnweagntf |
| 421 | tcsvlheglh nhhtekslsh spgk |
| SEQ ID NO: 30 | |
| 1 | diqmtqspss lsasvgdrvt itcrtsenlh nylawyqqkp gkspklliyd aktladgvps |
| 61 | rfsgsgsgtd ytltisslqp edfatyycqh fwsdpytfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |
| SEQ ID NO: 38 | |
| 1 | evllqqsgpe lvkpgasvki pckasgytft dynmdwvkqs hgkslewigq inpnnggiff |
| 61 | nqkfkgkatl tvdkssntaf mevrsltsed tavyycarea ittvgamdyw gqgtsvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 39 | |
| 1 | qvalvqsgae vkkpgasvkv sckasgytft dynmdwvrqa pgkslewigq inpnnggiff |
| 61 | nqkfkgratl tvdtstntay melrslrsdd tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 40 | |
| 1 | qvqlvqsgae vkkpgssvkv sckasgytft dynmdwvrqa pgkslewigq inpnnggiff |
| 61 | nqkfkgratl tvdkstntay melsslrsed tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 41 | |
| 1 | qvqlvqsgae vkkpgasvkv sckasgytft dynmdwvrqa pggglewmgq inpnnggiff |
| 61 | nqkfkgrvtl ttdtststay melrslrsdd tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 43 | |
| 1 | qvqlvqsgae vkkpgasvkv sckasgytft dynmdwvrqa pgqslewmgq inpnnggiff |
| 61 | nqkfqgrvtl ttdtststay melrslrsdd tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreegyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 42 | |
| 1 | qvqlvqsgae vkkpgasvkv sckasgytft dynmdwvrqa pgqglewmgq inpnnggiff |
| 61 | nqkfqgrvtl ttdtststay melrslrsdd tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 44 | |
| 1 | qvqlvqsgae vkkpgssvkv sckasgytfs dynmdwvrga pgqglewmgq inpnnggiff |
| 61 | nqkfkgrvtl tadkststay melsslrsed tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 45 | |
| 1 | qvqlvqsgae vkkpgssvkv sckasgytfs dynmdwvrqa pggglewmgq inpnnggiff |
| 61 | nqkfqgrvtl tadkststay melsslrsed tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 46 | |
| 1 | qvqlvqsgae vkkpgasvkv sckasgytft dynmdwvrqa pggslewmgq inpynhliff |
| 61 | ngkfqgrvtl ttdtststay melrslrsdd tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 47 | |
| 1 | qvqlvqsgae vkkpgasvkv sckasgytft dynmdwvrga pgqslewmgq inpnngliff |
| 61 | nqkfqgrvtl ttdtststay melrslrsdd tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 48 | |
| 1 | qvqlvqsgae vkkpgssvkv sckasgytfs dynmdwvrga pgqglewmgq inpnngliff |
| 61 | nqkfkgrvtl tadkststay melsslrsed tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreegyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 49 | |
| 1 | qvqlvqsgae vkkpgssvkv sckasgytfs dynmdwvrqa pgqglewmgq inpynhliff |
| 61 | nqkfkgrvtl tadkststay melsslrsed tavyycarea ittvgamdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 38 | |
| 1 | evllqqsgpe lvkpgasvki pckasgytft dynmdwvkqs hgkslewigq inpnnggiff |
| 61 | nqkfkgkatl tvdkssntaf mevrsltsed tavyycarea ittvgamdyw gqgtsvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflippkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| SEQ ID NO: 51 | |
| 1 | qvtlkesgpa lvkptqtltl tctfsgfsln tygmgvswir qppgkalewl ahiywdddkr |
| 61 | ynpslktrlt iskdtsknqv vltitnvdpv dtavyycagr gyddywgywg qgtlvtissa |
| 121 | stkgpsvfpl apsskstsgg taalgclvkd yfpepvtvsw nsgaltsgvh tfpavlqssg |
| 181 | lyslssvvtv pssslgtqty icnvnhkpsn tkvdkrvepk scdkthtopp cpapellggp |
| 241 | svflfppkpk dtlmisrtpe vtcvvvdvsh edpevkfnwy vdgvevhnak tkpreegyns |
| 301 | tyrvvsvltv lhqdwlngke ykckvsnkal papiektisk akgqprepqv ytlppsreem |
| 361 | tknqvsltcl vkgfypsdia vewesngqpe nnykttppvl dsdgsfflys kltvdksrwq |
| 421 | qgnvfscsvm healhnhytq kslslspgk |
| SEQ ID NO: 52 | |
| 1 | qvtlkesgpt lvkptqtltl tctfsgfsln tygmgvswir qppgkglewl ahiywdddkr |
| 61 | ynpslksrlt itkdtsknqv vltitnmdpv dtatyycagr gyddywgywg qgtlvtvssa |
| 121 | stkgpsvfpl apsskstsgg taalgclvkd yfpepvtvsw nsgaltsgvh tfpavlqssg |
| 181 | lyslssvvtv pssslgtqty icnvnhkpsn tkvdkrvepk scdkthtopp cpapellggp |
| 241 | svflfppkpk dtlmisrtpe vtcvvvdvsh edpevkfnwy vdgvevhnak tkpreegyns |
| 301 | tyrvvsvltv lhqdwlngke ykckvsnkal papiektisk akgqprepqv ytlppsreem |
| 361 | tknqvsltcl vkgfypsdia vewesngqpe nnykttppvl dsdgsfflys kltvdksrwq |
| 421 | qgnvfscsvm healhnhytq kslslspgk |
| SEQ ID NO: 54 | |
| 1 | qvtlkesgpg ilqpsqtlsl tcsfsgfsls tygmgvgwir qpsgkglewl adiwwdddky |
| 61 | ynpslksrlt iskdtssnev flkiaivdta dtatyycarr ghysamdywg qgtsvtvssa |
| 121 | stkgpsvfpl apsskstsgg taalgclvkd yfpepvtvsw nsgaltsgvh tfpavlqssg |
| 181 | lyslssvvtv pssslgtqty icnvnhkpsn tkvdkrvepk scdkthtopp cpapellggp |
| 241 | svflfppkpk dtlmisrtpe vtcvvvdvsh edpevkfnwy vdgvevhnak tkpreegyns |
| 301 | tyrvvsvltv lhqdwingke ykckvsnkal papiektisk akgqprepqv ytlppsreem |
| 361 | tknqvsltcl vkgfypsdia vewesngqpe nnykttppvl dsdgsfflys kltvdksrwq |
| 421 | qgnvfscsvm healhnhytq kslslspgk |
| SEQ ID NO: 55 | |
| 1 | qvtlkesgpg ilgpsqtlsl tcsfsgfsln tygmgvswir qpsgkglewl ahiywdddkr |
| 61 | ynpslksrlt iskdasnnrv flkitsvdta dtatyycagr gyddywgywg qgtlvtisaa |
| 121 | stkgpsvfpl apsskstsgg taalgclvkd yfpepvtvsw nsgaltsgvh tfpavlqssg |
| 181 | lyslssvvtv pssslgtqty icnvnhkpsn tkvdkrvepk scdkthtopp cpapellggp |
| 241 | svflfppkpk dtlmisrtpe vtcvvvdvsh edpevkfnwy vdgvevhnak tkpreegyns |
| 301 | tyrvvsvltv lhqdwingke ykckvsnkal papiektisk akgqprepqv ytlppsreem |
| 361 | tknqvsltcl vkgfypsdia vewesngqpe nnykttppvl dsdgsfflys kltvdksrwq |
| 421 | qgnvfscsvm healhnhytq kslslspgk |
| SEQ ID NO: 56 | |
| 1 | gitlkesgpt lvkptqtltl tctfsgfsls tygmgvgwir qppgkalewl adiwwdddky |
| 61 | ynpslksrlt itkdtsknqv vltmtnmdpv dtatyycarr ghysamdywg qgtlvtvssa |
| 121 | stkgpsvfpl apsskstsgg taalgclvkd yfpepvtvsw nsgaltsgvh tfpavlqssg |
| 181 | lyslssvvtv pssslgtqty icnvnhkpsn tkvdkrvepk scdkthtopp cpapellggp |
| 241 | svflfppkpk dtlmisrtpe vtcvvvdvsh edpevkfnwy vdgvevhnak tkpreegyns |
| 301 | tyrvvsvltv lhqdwingke ykckvsnkal papiektisk akgqprepqv ytlppsreem |
| 361 | tknqvsltcl vkgfypsdia vewesngqpe nnykttppvl dsdgsfflys kltvdksrwq |
| 421 | nvfscsvm healhnhytq kslslspgk |
| SEQ ID NO: 57 | |
| 1 | qvtlkesgpa lvkptqtltl tctfsgfsls tygmgvgwir qppgkalewl adiwwdddky |
| 61 | ynpslksrlt iskdtsknqv vltmtnmdpv dtavyycarr ghysamdywg qgtlvtvssa |
| 121 | stkgpsvfpl apsskstsgg taalgclvkd yfpepvtvsw nsgaltsgvh tfpavlqssg |
| 181 | lyslssvvtv pssslgtqty icnvnhkpsn tkvdkrvepk scdkthtopp cpapellggp |
| 241 | svflfppkpk dtlmisrtpe vtcvvvdvsh edpevkfnwy vdgvevhnak tkpreegyns |
| 301 | tyrvvsvltv lhqdwlngke ykckvsnkal papiektisk akgqprepqv ytlppsreem |
| 361 | tknqvsltcl vkgfypsdia vewesngqpe nnykttppvl dsdgsfflys kltvdksrwq |
| 421 | qgnvfscsvm healhnhytq kslslspgk |
| SEQ ID NO: 50 | |
| 1 | gvtlkesgpg ilgpsqtlsl tcsfsgfsln tygmgvswir qpsgkglewl ahiywdddkr |
| 61 | ynpslksrlt iskdasnnrv flkitsvdta dtatyycagr gyddywgywg qgtlvtisaa |
| 121 | kttppsvypl apgsaaqtns mvtlgclvkg yfpepvtvtw nsgslssgvh tfpavlqsdl |
| 181 | ytlsssvtvp sstwpsetvt cnvahpasst kvdkkivprd cgckpcictv pevssvfifp |
| 241 | pkpkdvltit ltpkvtcvvv diskddpevq fswfvddvev htaqtqpree qfnstfrsvs |
| 301 | elpimhqdwl ngkefkcrvn saafpapiek tisktkgrpk apqvytippp keqmakdkvs |
| 361 | ltcmitdffp editvewqwn gqpaenyknt qpimdtdgsy fvysklnvqk snweagntft |
| 421 | csvlheglhn hhtekslshs pgk |
| SEQ ID NO: 31 | |
| 1 | divmtqsqkf mstsvgdrvs vtckasqnvg tnvawfqqkp gaspkaliys asyrysgvpd |
| 61 | rftgsgsgtd filtisnvqs edlaeyfcqg ynnypltfga gtklelkrad aaptvsifpp |
| 121 | sseqltsgga svvcflnnfy pkdinvkwki dgserqngvl nswtdqdskd stysmsstlt |
| 181 | ltkdeyerhn sytceathkt stspivksfn rnec |
| SEQ ID NO: 53 | |
| 1 | qvtlkesgpg ilqpsqtlsl tcsfsgfsls tygmgvgwir qpsgkglewl adiwwdddky |
| 61 | ynpslksrlt iskdtssnev flkiaivdta dtatyycarr ghysamdywg qgtsvtvssa |
| 121 | kttppsvypl apgsaaqtns mvtlgclvkg yfpepvtvtw nsgslssgvh tfpavlqsdl |
| 181 | ytlsssvtvp sstwpsetvt cnvahpasst kvdkkivprd cgckpcictv pevssvfifp |
| 241 | pkpkdvltit ltpkvtcvvv diskddpevq fswfvddvev htaqtqpree qfnstfrsvs |
| 301 | elpimhqdwl ngkefkcrvn saafpapiek tisktkgrpk apqvytippp keqmakdkvs |
| 361 | ltcmitdffp editvewqwn gqpaenyknt qpimdtdgsy fvysklnvqk snweagntft |
| 421 | csvlheglhn hhtekslshs pgk |
| SEQ ID NO: 34 | |
| 1 | divmtqsqkf mstsvgdrvs vtckasqnvg tnvawyqqkp gospkaliys psyrysgvpd |
| 61 | rftgsgsgtd ftltisnvqs edlaeyfcqg ynsyphtfgg gtklemkrad aaptvsifpp |
| 121 | sseqltsgga svvcflnnfy pkdinvkwki dgserqngvl nswtdqdskd stysmsstlt |
| 181 | ltkdeyerhn sytceathkt stspivksfn rnec |

Exemplary anti-GDF15 antibodies useful in the methods and compositions of the invention may, for example, include a heavy chain variable region comprising any one of the two sets of CDR_{H1}, CDR_{H2}, and CDR_{H3} region sequences set forth in Table 4 below.

TABLE 4

| | CDR_{H1} | CDR_{H2} | CDR_{H3} |
|---|---|---|---|
| 1 | GYTFSSYNID | GINPIFGTAFYNQKFQG | EAITTVGAMDH |
| | (SEQ ID NO: 58) | (SEQ ID NO: 59) | (SEQ ID NO: 60) |
| 2 | GYTFSSYNID | GNPIFGTAFYNQKFQG | EAITTVGAMDH |
| | (SEQ ID NO: 58) | (SEQ ID NO: 108) | (SEQ ID NO: 60) |

Exemplary anti-GDF15 antibodies useful in the methods and compositions of the invention may, for example, include a light chain variable region comprising the CDR_{L1}, CDR_{L2}, and CDR_{L3} region sequences set forth in Table 5 below.

TABLE 5

| | CDRL1 | CDRL2 | CDRL3 |
|---|---|---|---|
| 1 | RTSQSVHNYLA | DASTRAD | QQFWSWPWT |
| | (SEQ ID NO: 61) | (SEQ ID NO: 62) | (SEQ ID NO: 63) |

Exemplary anti-GDF15 antibodies useful in the methods and compositions of the invention may, for example, include (i) any one of the two sets of CDR_{H1}, CDR_{H2}, and CDR_{H3} region sequences set forth in Table 4, and (ii) the CDR_{L1}, CDR_{L2}, and CDR_{L3} region sequences set forth in Table 5. For example, an exemplary anti-GDF15 antibody may comprise a CDR_{H1} comprising the amino acid sequence of SEQ ID NO: 58, a CDR_{H2} comprising the amino acid sequence of SEQ ID NO: 59, a CDR_{H3}, comprising the amino acid sequence of SEQ ID NO: 60, a CDR_{L1} comprising the amino acid sequence of SEQ ID NO: 61, a CDR_{L2} comprising the amino acid sequence of SEQ ID NO: 62, and a CDR_{L3} comprising the amino acid sequence of SEQ ID NO: 63. Another exemplary anti-GDF15 antibody may comprise a CDR_{H1} comprising the amino acid sequence of SEQ ID NO: 58, a CDR_{H2} comprising the amino acid sequence of SEQ ID NO: 108, a CDR_{H3}, comprising the amino acid sequence of SEQ ID NO: 60, a CDR_{L1} comprising the amino acid sequence of SEQ ID NO: 61, a CDR_{L2} comprising the amino acid sequence of SEQ ID NO: 62, and a CDR_{L3} comprising the amino acid sequence of SEQ ID NO: 63.

Exemplary anti-GDF-15 antibodies useful in the practice of the invention are described in U.S. Patent Application Publication No. 2020/0055930 (the disclosure of which is incorporated by reference herein for all purposes) including GDF15_001, as well as human or humanized forms thereof.

Exemplary anti-GDF15 antibodies useful in the methods and compositions of the invention may, for example, include any one of the 22 sets of heavy chain variable region and light chain variable region sequences set forth in Table 6 below, or any of the two sets of heavy chain and light chain sequences set forth in Table 7 below.

TABLE 6

| | Heavy Chain Variable | Light Chain Variable |
|---|---|---|
| | Region | Region |
| 1 | SEQ ID NO: 64 | SEQ ID NO: 85 |
| 2 | SEQ ID NO: 65 | SEQ ID NO: 86 |
| 3 | SEQ ID NO: 66 | SEQ ID NO: 87 |
| 4 | SEQ ID NO: 67 | SEQ ID NO: 88 |
| 5 | SEQ ID NO: 68 | SEQ ID NO: 89 |
| 6 | SEQ ID NO: 69 | SEQ ID NO: 90 |
| 7 | SEQ ID NO: 70 | SEQ ID NO: 91 |
| 8 | SEQ ID NO: 71 | SEQ ID NO: 92 |
| 9 | SEQ ID NO: 72 | SEQ ID NO: 93 |
| 10 | SEQ ID NO: 73 | SEQ ID NO: 94 |
| 11 | SEQ ID NO: 74 | SEQ ID NO: 95 |
| 12 | SEQ ID NO: 75 | SEQ ID NO: 96 |
| 13 | SEQ ID NO: 76 | SEQ ID NO: 97 |
| 14 | SEQ ID NO: 77 | SEQ ID NO: 98 |
| 15 | SEQ ID NO: 78 | SEQ ID NO: 99 |
| 16 | SEQ ID NO: 79 | SEQ ID NO: 100 |
| 17 | SEQ ID NO: 80 | SEQ ID NO: 101 |
| 18 | SEQ ID NO: 81 | SEQ ID NO: 102 |
| 19 | SEQ ID NO: 82 | SEQ ID NO: 103 |
| 20 | SEQ ID NO: 83 | SEQ ID NO: 104 |
| 21 | SEQ ID NO: 84 | SEQ ID NO: 105 |
| 22 | SEQ ID NO: 109 | SEQ ID NO: 85 |

TABLE 7

| | Heavy Chain | Light Chain |
|---|---|---|
| 1 | SEQ ID NO: 106 | SEQ ID NO: 107 |
| 2 | SEQ ID NO: 110 | SEQ ID NO: 107 |

SEQ ID NO: 64
QVQLVQSGAEVKKPGSSVKVSCKASGYTFSSYNISWVRQAPGQGLEWMGGINPINGLAFYNQKFQGRVTITAD
ESTSTAYMELSSLRSEDTAVYYCAREAITTVGAMDYWGQGTLVTVSS
SEQ ID NO: 65
QVQLVQSGAEVKKPGSSVKVSCKASGYTFSSYNIDWVRQAPGQGLEWMGQINPNNGLAFYNQKFQGRVTITAD
ESTSTAYMELSSLRSEDTAVYYCAREAITTVGAMDYWGQGTLVTVSS
SEQ ID NO: 66
QVQLVQSGAEVKKPGSSVKVSCKASGYTFSDYNIDWVRQAPGQGLEWMGQINPNNGLIFFNQKFQGRVTITAD
ESTSTAYMELSSLRSEDTAVYYCAREVITTVGAMDYWGQGTLVTVSS
SEQ ID NO: 67
QVQLVQSGAEVKKPGSSVKVSCKASGYTFSSYNIDWVRQAPGQGLEWMGGINPINGLIFFNQKFQGRVTITAD
ESTSTAYMELSSLRSEDTAVYYCAREAITTVGAMDHWGQGTLVTVSS
SEQ ID NO: 68
QVQLVQSGAEVKKPGSSVKVSCKASGYTFSDYNMDWVRQAPGQGLEWMGQINPNNGLANYNQKFQGRVTITAD
ESTSTAYMELSSLRSEDTAVYYCAREAITTVGAMDYWGQGTLVTVSS
SEQ ID NO: 69
QVQLVQSGAEVKKPGSSVKVSCKASGYTFTDYNIDWVRQAPGQGLEWMGQINPNNGLIFYNQKFQGRVTITAD
ESTSTAYMELSSLRSEDTAVYYCAREAITTVGAMDYWGQGTLVTVSS
SEQ ID NO: 70
QVQLVQSGAEVKKPGSSVKVSCKASGYTFTDYNIDWVRQAPGQGLEWMGQINPNNGLAFYNQKFQGRVTITAD
ESTSTAYMELSSLRSEDNAVYYCAREAITTVGAMDYWGQGTLVTVSS
SEQ ID NO: 71
QVQLVQSGAEVKKPGSSVKVSCKASGYTFSDYNMDWVRQAPGQGLEWMGQINPNNGLAFYNQKFQGRVTITAD
ESTSTAYMELSSLRSEDTAVYYCAREAITTVGATDYWGQGTLVTVSS
SEQ ID NO: 72
QVQLVQSGAEVKKPGSSVKVSCKASGYTFSSYNIDWVRQAPGQGLEWMGQINPINGLAFYNQKFQGRVTITAD
ESTSTAYMELSSLRSEDTAVYYCAREAITTVGAMDYWGQGTLVTVSS
SEQ ID NO: 73
QVQLVQSGAEVKKPGSSVKVSCKASGYTFSDYNMDWVRQAPGQGLEWMGGINPNNGLAFYNQKFQGRVTITAD
ESTSTAYMELSSLRSEDTAVYYCAREAITTVGAMDYWGQGTLVTVSS
SEQ ID NO: 74
QVQLVQSGAEVKKPGSSVKVSCKASGYTFSDYNMDWVRQAPGQGLEWMGQINPIFGLAFYAQKFQGRVTITAD
ESTSTAYMELSSLRSEDTAVYYCAREVITTVGAMDYWGQGTLVTVSS
SEQ ID NO: 75
QVQLVQSGAEVKKPGSSVKVSCKASGYTFSDYNIDWVRQAPGQGLEWMGGINPNNGLAFFNQKFQGRVTITAD
ESTSTAYMELSSLRSEDTAVYYCAREAITTVGAMDYWGQGTLVTVSS
SEQ ID NO: 76
QVQLVQSGAEVKKPGSSVKVSCKASGYTFSSYNISWVRQAPGQGLEWMGQINPNNGLAFYNQKFQGRVTITAD
ESTSTAYMELSSLRSEDTAVYYCAREAITTVGAMEYWGQGTLVTVSS
SEQ ID NO: 77
QVQLVQSGAEVKKPGSSVKVSCKASGYTFTSYNISWVRQAPGQGLEWMGQINPNNGLIFFAQKFQGRVTITAD
ESTSTAYMELSSLRSEDTAVYYCAREAITTVGAMDQWGQGTLVTVSS
SEQ ID NO: 78
QVQLVQSGAEVKKPGSSVKVSCKASGYTFSDYNISWVRQAPGQGLEWMGGINPIFGLAFYNQKFQGRVTITAD
ESTSTAYMELSSLRSEDTAVYYCAREAITTVGAMDYWGQGTLVTVSS
SEQ ID NO: 79
QVQLVQSGAEVKKPGSSVKVSCKASGYTFTDYNISWVRQAPGQGLEWMGQINPNNGLAFYAQKFQGRVTITAD
ESTSTAYMELSSLRSEDTAVYYCAREFITTVGAMDYWGQGTLVTVSS
SEQ ID NO: 80
QVQLVQSGAEVKKPGSSVKVSCKASGYTFSDYNMDWVRQAPGQGLEWMGGINPNNGTAFYAQKFQGRVTITAD
ESTSTAYMELSSLRSEDTAVYYCAREAITTVGAMDQWGQGTLVTVSS
SEQ ID NO: 81
QVQLVQSGAEVKKPGSSVKVSCKASGYTFSSYNIDWVRQAPGQGLEWMGQINPNNGLANYAQKFQGRVTITAD
ESTSTAYMELSSLRSEDTAVYYCAREAITTIGAMDYWGQGTLVTVSS
SEQ ID NO: 82
QVQLVQSGAEVKKPGSSVKVSCKASGYTFTSYNIDWVRQAPGQGLEWMGQINPNNGLAFYNQKFQGRVTITAD
ESTSTAYMELSSLRSEDTAVYYCAREQITTVGAMDYWGQGTLVTVSS
SEQ ID NO: 83
QVQLVQSGAEVKKPGSSVKVSCKASGYTFSSYNIDWVRQAPGQGLEWMGGINPIFGLAFYNQKFQGRVTITAD
ESTSTAYMELSSLRSEDTAVYYCAREAITTVGAMDPWGQGTLVTVSS
SEQ ID NO: 84
QVQLVQSGAEVKKPGSSVKVSCKASGYTFSSYNIDWVRQAPGQGLEWMGGINPIFGTAFYNQKFQGRVTITAD
ESTSTAYMELSSLRSEDTAVYYCAREAITTVGAMDHWGQGTLVTVSS
SEQ ID NO: 85
EIVLTQSPATLSLSPGERATLSCRASQSVHSYLAWYQQKPGQAPRLLIYDASNRATGIPARESGSGSGTDFTL
TISSLEPEDFAVYYCQQFWSWPWTFGQGTKVEIK
SEQ ID NO: 86
EIVLTQSPATLSLSPGERATLSCRTSQNVHSYLAWYQQKPGQAPRLLIYDASTRADGIPARFSGSGSGTDFTL
TISSLEPEDFAVYYCQQFWSDPWTFGQGTKVEIK
SEQ ID NO: 87
EIVLTQSPATLSLSPGERATLSCRTSQSVHSYLAWYQQKPGQAPRLLIYDAKTRADGIPARESGSGSGTDFTL
TISSLEPEDFAVYYCQQFSSDPYTFGQGTKVEIK
SEQ ID NO: 88
EIVLTQSPATLSLSPGERATLSCRTSENVHSYLAWYQQKPGQAPRLLIYDASNLADGIPARFSGSGSGTDFTL
TISSLEPEDFAVYYCQQFWSDPYTFGQGTKVEIK
SEQ ID NO: 89
EIVLTQSPATLSLSPGERATLSCRASQNLHSYLAWYQQKPGQAPRLLIYDASTRADGIPARFSGSGSGTDFTL
TISSLEPEDFAVYYCQQFWSDPYTFGQGTKVEIK
SEQ ID NO: 90
EIVLTQSPATLSLSPGERATLSCRASQNVHSYLAWYQQKPGQAPRLLIYDASTRADGIPARESGSGSGTDFTL
TISSLEPEDFAVYYCQQFWNDPYTFGQGTKVEIK
SEQ ID NO: 91
EIVLTQSPATLSLSPGERATLSCRTSQSVHSYLAWYQQKPGQAPRLLIYDAKTRATGIPAIRESGSGSGTDFT
LTISSLEPEDFAVYYCQQFSSDPYTFGQGTKVEIK
SEQ ID NO: 92
EIVLTQSPATLSLSPGERATLSCRTSQNVHSYLAWYQQKPGQAPRLLIYDASNLADGIPARESGSGSGTDFTL
TISSLEPEDFAVYYCQQFSNDPWTFGQGTKVEIK
SEQ ID NO: 93
EIVLTQSPATLSLSPGERATLSCRTSQNVHNYLAWYQQKPGQAPRLLIYDASNRATGIPARFSGSGSGTDFTL
TISSLEPEDFAVYYCQQFWSDPYTFGQGTKVEIK
SEQ ID NO: 94
EIVLTQSPATLSLSPGERATLSCRTSESVHSYLAWYQQKPGQAPRLLIYDASNRATGIPARFSGSGSGTDFTL
TISSLEPEDFAVYYCQQFWNWPWTFGQGTKVEIK
SEQ ID NO: 95
EIVLTQSPATLSLSPGERATLSCRTSQSVHNYLAWYQQKPGQAPRLLIYDASTRADGIPARFSGSGSGTDFTL
TISSLEPEDFAVYYCQQFSSDPYTFGQGTKVEIK
SEQ ID NO: 96
EIVLTQSPATLSLSPGERATLSCRTSQSLHSYLAWYQQKPGQAPRLLIYDASNRATGIPARESGSGSGTDFTL
TISSLEPEDFAVYYCQQFWNDPWTFGQGTKVEIK
SEQ ID NO: 97
EIVLTQSPATLSLSPGERATLSCRTSQNVHSYLAWYQQKPGQAPRLLIYDAKNRADGIPARESGSGSGTDFTL
TISSLEPEDFAVYYCQQFWSDPYTFGQGTKVEIK
SEQ ID NO: 98
EIVLTQSPATLSLSPGERATLSCRTSQNVHSYLAWYQQKPGQAPRLLIYDASNRADGIPARFSGSGSGTDFTL
TISSLEPEDFAVYYCQQFWNDPYTFGQGTKVEIK
SEQ ID NO: 99
EIVLTQSPATLSLSPGERATLSCRTSENVHSYLAWYQQKPGQAPRLLIYDASTLATGIPARFSGSGSGTDFTL
TISSLEPEDFAVYYCQQFWSWPWTFGQGTKVEIK
SEQ ID NO: 100
EIVLTQSPATLSLSPGERATLSCRTSQSVSNYLAWYQQKPGQAPRLLIYDAKNRATGIPARFSGSGSGTDFTL
TISSLEPEDFAVYYCQQFWNDPWTFGQGTKVEIK
SEQ ID NO: 101
EIVLTQSPATLSLSPGERATLSCRTSESVSSYLAWYQQKPGQAPRLLIYDAKTRADGIPARFSGSGSGTDFTL
TISSLEPEDFAVYYCQQFWSWPWTFGQGTKVEIK
SEQ ID NO: 102
EIVLTQSPATLSLSPGERATLSCRTSESVHSYLAWYQQKPGQAPRLLIYDASTRADGIPARFSGSGSGTDFTL
TISSLEPEDFAVYYCQQFWSDPYTFGQGTKVEIK
SEQ ID NO: 103
EIVLTQSPATLSLSPGERATLSCRASQSLSSYLAWYQQKPGQAPRLLIYDAKNRADGIPARFSGSGSGTDFTL
TISSLEPEDFAVYYCQQFSSDPYTFGQGTKVEIK
SEQ ID NO: 104
EIVLTQSPATLSLSPGERATLSCRASQNVHNYLAWYQQKPGQAPRLLIYDASNRADGIPARFSGSGSGTDFTL
TISSLEPEDFAVYYCQQFWSWPWTFGQGTKVEIK
SEQ ID NO: 105
EIVLTQSPATLSLSPGERATLSCRTSQSVHNYLAWYQQKPGQAPRLLIYDASTRADGIPARFSGSGSGTDFTL
TISSLEPEDFAVYYCQQFWSWPWTFGQGTKVEIK
SEQ ID NO: 106
QVQLVQSGAEVKKPGSSVKVSCKASGYTFSSYNIDWVRQAPGQGLEWMGGINPIFGTAFYNQKFQGRVTITAD
ESTSTAYMELSSLRSEDTAVYYCAREAITTVGAMDHWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVE
PKSCDKTHTCPPCPAPEAAGAPSVELFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCSVMHEALHNH
YTQKSLSLSPG
SEQ ID NO: 107
EIVLTQSPATLSLSPGERATLSCRTSQSVHNYLAWYQQKPGQAPRLLIYDASTRADGIPARFSGSGSGTDFTL
TISSLEPEDFAVYYCQQFWSWPWTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
SEQ ID NO: 109
QVQLVQSGAEVKKPGSSVKVSCKASGYTFSSYNIDWVRQAPGQGLEWMGGNPIFGTAFYNQKFQGRVTITADE
STSTAYMELSSLRSEDTAVYYCAREAITTVGAMDHWGQGTLVTVSS
SEQ ID NO: 110
QVQLVQSGAEVKKPGSSVKVSCKASGYTFSSYNIDWVRQAPGQGLEWMGGNPIFGTAFYNQKFQGRVTITADE
STSTAYMELSSLRSEDTAVYYCAREAITTVGAMDHWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEP
KSCDKTHTCPPCPAPEAAGAPSVELFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVFSCSVMHEALHNHY
TQKSLSLSPG

An exemplary anti-GDF-15 antibody is PF-06946860 (Ponsegromab).

Exemplary anti-GDF15 antibodies useful in the methods and compositions of the invention may, for example, include a heavy chain variable region comprising the CDR_{H1}, CDR_{H2}, and CDR_{H3} region sequences set forth in Table 8 below.

TABLE 8

| | CDR_{H1} | CDR_{H2} | CDR_{H3} |
|---|---|---|---|
| 1 | DYWIS | IIDPSGSYTIYSPSFQG | VSYYGGYFDI |
| | (SEQ ID NO: 111) | (SEQ ID NO: 112) | (SEQ ID NO: 113) |

Exemplary anti-GDF15 antibodies useful in the methods and compositions of the invention may, for example, include a light chain variable region comprising the CDR_{L1}, CDR_{L2}, and CDR_{L3} region sequences set forth in Table 9 below.

TABLE 9

| | CDRL₁ | CDRL₂ | CDRL₃ |
|---|---|---|---|
| 1 | RASQSISNNLN | AASNLQS | FQLDHSPFT |
| | (SEQ ID NO: 114) | (SEQ ID NO: 115) | (SEQ ID NO: 116) |

Exemplary anti-GDF15 antibodies useful in the methods and compositions of the invention may, for example, include (i) the CDR_{H1}, CDR_{H2}, and CDR_{H3} region sequences set forth in Table 8, and (ii) the CDR_{L1}, CDR_{L2}, and CDR_{L3} region sequences set forth in Table 9. For example, an exemplary anti-GDF15 antibody may comprise a CDR_{H1} comprising the amino acid sequence of SEQ ID NO: 111, a CDR_{H2} comprising the amino acid sequence of SEQ ID NO: 112, a CDR_{H3}, comprising the amino acid sequence of SEQ ID NO: 113, a CDR_{L1} comprising the amino acid sequence of SEQ ID NO: 114, a CDR_{L2} comprising the amino acid sequence of SEQ ID NO: 115, and a CDR_{L3} comprising the amino acid sequence of SEQ ID NO: 116.

Exemplary anti-GDF-15 antibodies useful in the practice of the invention are described in U.S. Patent Application Publication No. 2019/0135908 (the disclosure of which is incorporated by reference herein for all purposes) including ABGDF15-A, ABGDF15-G, ABGDF15-B, ABGDF15-C, ABGDF15-F, ABGDF15-E, and ABGDF15-D, as well as human or humanized forms thereof.

Exemplary anti-GDF15 antibodies useful in the methods and compositions of the invention may, for example, include any one of the seven sets of heavy chain variable region and light chain variable region sequences set forth in Table 10 below, or the heavy chain and light chain sequences set forth in Table 11 below.

TABLE 10

| | Heavy Chain Variable | Light Chain Variable |
|---|---|---|
| | Region | Region |
| 1 | SEQ ID NO: 117 | SEQ ID NO: 124 |
| 2 | SEQ ID NO: 118 | SEQ ID NO: 125 |
| 3 | SEQ ID NO: 119 | SEQ ID NO: 126 |
| 4 | SEQ ID NO: 120 | SEQ ID NO: 127 |
| 5 | SEQ ID NO: 121 | SEQ ID NO: 128 |
| 6 | SEQ ID NO: 122 | SEQ ID NO: 129 |
| 7 | SEQ ID NO: 123 | SEQ ID NO: 130 |

TABLE 11

| Antibody Name | Heavy Chain | Light Chain |
|---|---|---|
| 8 | SEQ ID NO: 131 | SEQ ID NO: 132 |

SEQ ID NO: 117
EVQLVQSGAEVKKPGESLKISCKGSGYSFTDYWISWVRQMPGKGLEWMGIIDPSGSYTIYSPSFQGQVTISAD
KSISTAYLOWSSLKASDTAMYYCARVSYYGGYFDIWGQGTLVTVSS
SEQ ID NO: 118
QVQLVQSGAEVKKPGSSVKVSCKASGGTFSSHYINWVRQAPGQGLEWMGGIIPAFGGANYAQKFQGRVTITAD
ESTSTAYMELSSLRSEDTAVYYCARFGSVYVSRYSSYYHMDVWGQGTLVTVSS
SEQ ID NO: 119
QVQLVQSGAEVKKPGSSVKVSCKASGGTFRSYAVSWVRQAPGQGLEWMGGIIPIFGTANYAQKFQGRVTITAD
ESTSTAYMELSSLRSEDTAVYYCARGPIIMGYQFGLFDHWGQGTLVTVSS
SEQ ID NO: 120
QVQLVQSGAEVKKPGSSVKVSCKASGGTFRSYAVSWVRQAPGQGLEWMGGIIPIFGTANYAQKFQGRVTITAD
ESTSTAYMELSSLRSEDTAVYYCARGPIIMGYQFGLFDHWGQGTLVTVSS
SEQ ID NO: 121
EVQLVQSGAEVKKPGESLKISCKGSGYSFTSYWIGWVRQMPGKGLEWMGVIDPDGSYTIYSPSFQGQVTISAD
KSISTAYLOWSSLKASDTAMYYCARYGRYGTYFDYWGQGTLVTVSS
SEQ ID NO: 122
EVQLVQSGAEVKKPGESLKISCKGSGYSFTSYWIGWVRQMPGKGLEWMGVIDPGGSYTIYSPSFQGQVTISAD
KSISTAYLOWSSLKASDTAMYYCARYGRYGTYFDYWGQGTLVTVSS
SEQ ID NO: 123
EVQLVQSGAEVKKPGESLKISCKGSGYSFTSYWIGWVRQMPGKGLEWMGVIDPSGSYTIYSPSFQGQVTISAD
KSISTAYLOWSSLKASDTAMYYCARYGRYGTYFDYWGQGTLVTVSS
SEQ ID NO: 124
DIQMTQSPSSLSASVGDRVTITCRASQSISNNLNWYQQKPGKAPKLLIYAASNLQSGVPSRESGSGSGTDETL
TISSLQPEDFATYYCFQLDHSPFTFGQGTKVEIK
SEQ ID NO: 125
DIQMTQSPSSLSASVGDRVTITCRASQTIYRSLAWYQQKPGKAPKLLIYGASILQSGVPSRESGSGSGTDETL
TISSLOPEDFATYYCLQRYTSPFTFGQGTKVEIK
SEQ ID NO: 126
SYELTQPLSVSVALGQTARITCSGDNIGSHIVSWYQQKPGQAPVLVIYDKSNRPSGIPERFSGSNSGNTATLT
ISRAQAGDEADYYCQTWDSIGSVVFGGGTKLTVL
SEQ ID NO: 127
SYELTQPPSVSVSPGQTASITCSGDNIGSHIVSWYQQKPGQSPVLVIYDKSNRPSGIPERFSGSNSGNTATLT
ISGTQAMDEADYYCQTWDSIGSVVFGGGTKLTVL
SEQ ID NO: 128
QSVLTQPPSVSGAPGQRVTISCSGSSSNIGVLYVNWYQQLPGTAPKLLIYSNDNRPSGVPDRESGSKSGTSAS
LAITGLQAEDEADYYCQSWDSSSNYVFGGGTKLTVL
SEQ ID NO: 129
QSVLTQPPSVSGAPGQRVTISCSGSSSNIGVLYVNWYQQLPGTAPKLLIYSNDNRPSGVPDRESGSKSGTSAS
LAITGLQAEDEADYYCQSWDSSSNYVFGGGTKLTVL
SEQ ID NO: 130
QSVLTQPPSVSGAPGQRVTISCSGSSSNIGVLYVNWYQQLPGTAPKLLIYSNDNRPSGVPDRESGSKSGTSAS
LAITGLQAEDEADYYCQSWDSSSNYVEGGGTKLTVL
SEQ ID NO: 131
EVQLVQSGAEVKKPGESLKISCKGSGYSFTDYWISWVRQMPGKGLEWMGIIDPSGSYTIYSPSFQGQVTISAD
KSISTAYLOWSSLKASDTAMYYCARVSYYGGYFDIWGQGTLVTVSSASTKGPSVFPLAPSSKSTSGGTAALGC
LVKDYFPEPVTVSWNSGALTSGVHTFPAVLOSSGLYSLSSVVTVPSSSLGTQTYICNVNHKPSNTKVDKRVEP
KSCDKTHTCPPCPAPELLGGPSVELFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVESCSVMHEALHNHY
TQKSLSLSPGK
SEQ ID NO: 132
DIQMTQSPSSLSASVGDRVTITCRASQSISNNLNWYQQKPGKAPKLLIYAASNLQSGVPSRESGSGSGTDETL
TISSLQPEDFATYYCFQLDHSPFTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSENRGEC

An additional exemplary anti-GDF15 antibody is polyclonal goat IgG anti-human GDF-15 antibody, product number AF957, made by R&D systems.

Exemplary anti-GDF15 receptor antibodies include anti GFRAL antibodies. GFRAL is a GDF15 receptor (See Mullican et al. (2017) Nature Medicine, 23:1150-1157; Emmerson et al. (2017) Nature Medicine, 23:1215-1219; Hu et al. (2017) Nature, 550:255-259; and Yang et al. (2017) Nature Medicine, 23:1158-1166, the disclosures of which are incorporated by reference herein for all purposes).

Exemplary anti-GFRAL antibodies useful in the methods and compositions of the invention may, for example, include a heavy chain variable region comprising any one of the six sets of CDR_{H1}, CDR_{H2}, and CDR_{H3} region sequences set forth in Table 12 below.

TABLE 12

| | CDR_{H1} | CDR_{H2} | CDR_{H3} |
|---|---|---|---|
| 1 | GYTFTDYGVI | WINTYTGEPTYADDLKG | RYGPEDIDY |
| | (SEQ ID NO: 133) | (SEQ ID NO: 134) | (SEQ ID NO: 135) |
| 2 | DYGVI | WINTYTGEPTYADDLKG | RYGPEDIDY |
| | (SEQ ID NO: 136) | (SEQ ID NO: 134) | (SEQ ID NO: 135) |
| 3 | GYTFTDY | TYTG | YGPEDID |
| | (SEQ ID NO: 137) | (SEQ ID NO: 138) | (SEQ ID NO: 139) |
| 4 | GYTFTDYG | INTYTGEP | ARRYGPEDIDY |
| | (SEQ ID NO: 140) | (SEQ ID NO: 141) | (SEQ ID NO: 142) |
| 5 | TDYGVI | WMGWINTYTGEPT | ARRYGPEDID |
| | (SEQ ID NO: 143) | (SEQ ID NO: 144) | (SEQ ID NO: 145) |
| 6 | GYTFTDYGVI | WINTYTGEPT | RYGPEDIDY |
| | (SEQ ID NO: 133) | (SEQ ID NO: 146) | (SEQ ID NO: 135) |

Exemplary anti-GFRAL antibodies useful in the methods and compositions of the invention may, for example, include a light chain variable region comprising any one of the four sets of CDR_{L1}, CDR_{L2}, and CDR_{L3} region sequences set forth in Table 13 below.

TABLE 15

| | CDRL₁ | CDRL₂ | CDRL₃ |
|---|---|---|---|
| 1 | RASESVDNYGISEMS | AASHQGS | LQSKEVPWT |
| | (SEQ ID NO: 147) | (SEQ ID NO: 148) | (SEQ ID NO: 149) |
| 2 | SESVDNYGISE | AAS | SKEVPW |
| | (SEQ ID NO: 150) | (SEQ ID NO: 151) | (SEQ ID NO: 152) |
| 3 | ESVDNYGISE | AAS | LQSKEVPWT |
| | (SEQ ID NO: 153) | (SEQ ID NO: 151) | (SEQ ID NO: 149) |
| 4 | DNYGISFMSWF | LLIYAASHQG | LQSKEVPW |
| | (SEQ ID NO: 154) | (SEQ ID NO: 155) | (SEQ ID NO: 156) |

Exemplary anti-GFRAL antibodies useful in the methods and compositions of the invention may, for example, include (i) any one of the six sets of CDR_{H1}, CDR_{H2}, and CDR_{H3} region sequences set forth in Table 12, and (ii) any one of the four sets of CDR_{L1}, CDR_{L2}, and CDR_{L3} region sequences set forth in Table 13. For example, an exemplary anti-GFRAL antibody may comprise: (i) a CDR_{H1} comprising the amino acid sequence of SEQ ID NO: 133, a CDR_{H2} comprising the amino acid sequence of SEQ ID NO: 134, a CDR_{H3}, comprising the amino acid sequence of SEQ ID NO: 135, a CDR_{L1} comprising the amino acid sequence of SEQ ID NO: 147, a CDR_{L2} comprising the amino acid sequence of SEQ ID NO: 148, and a CDR_{L3} comprising the amino acid sequence of SEQ ID NO: 149.

Exemplary anti-GFRAL antibodies useful in the practice of the invention are described in U.S. Pat. No. 10,174,119 (the disclosure of which is incorporated by reference herein for all purposes) including 1C1, 3P10, 12A3, 5F12, 5A20, 8D8, 17J16, 25M22, 2B8, 22N5, 2123, 6N16, 1B3, 19K19, 2B3, 8C10, 2A9, 24G2, 6G9, 2B11, 1A3, P1B6, P1H8, or P8G4, as well as human or humanized forms thereof.

Exemplary anti-GFRAL antibodies useful in the methods and compositions of the invention may, for example, include any one of the two sets of heavy chain variable region and light chain variable region sequences set forth in Table 14.

TABLE 14

| | Heavy Chain Variable | Light Chain Variable |
|---|---|---|
| | Region | Region |
| 1 | SEQ ID NO: 157 | SEQ ID NO: 158 |
| 2 | SEQ ID NO: 159 | SEQ ID NO: 160 |

SEQ ID NO: 157
QIQLVQSGPELKKPGETVKISCKASGYTFTDYGVIWVKQAPGKALKWMGWINTYTGEPTYADDLKGRFAFSLE
TSASSASLQINNLKNEDTATYFCARRYGPEDIDYWGQGTTLTVSS
SEQ ID NO: 158
DIVLTQSPVSLAVSLGQRATISCRASESVDNYGISFMSWFQQKPGQPPKLLIYAASHQGSGVPARFSGSGSGT
DFSLNIHPMEEDDSAMYFCLQSKEVPWTFGGGTKLEIK
SEQ ID NO: 159
QVQLVQSGAEVKKPGSSVKVSCKASGYTFTDYGVIWVRQAPGQGLEWMGWINTYTGEPTYADDLKGRVTFTAD
ESTSTAYMELSSLRSEDTAVYYCARRYGPEDIDYWGQGTTVTVSS
SEQ ID NO: 160
DVVLTQSPLSLPVTLGQPASISCRASESVDNYGISFMSWFQQRPGQSPRLLIYAASHQGSGVPDRFSGSGSGT
DFTLKISRVEAEDVGVYFCLQSKEVPWTFGGGTKVEIK

An exemplary anti-GFRAL antibody is NGM120.

It is understood that the antibodies described herein can be designed, tested, and formulated using techniques known in the art.

In the present invention, among these, an anti-GDF15 antibody containing the GDF15-binding fragment described above or the like or an antagonist of a GDF-specific receptor is preferable as the substance inhibiting the action of GDF15, and an anti-GDF15 antibody is more preferable.

The antibody may be a neutralizing antibody, which reduces GDF15 activity. For example, the antibody may reduce GDF15 activity in an in vivo assay (see, e.g., Johnen et al., 2007, NATURE MEDICINE 13:1333-1340) by at least 10%, preferably 20%, 30% or 40%, and more preferably at least about 50%, 60%, 80% or 90% of GDF15 compared to GDF15 activity measured in the same assay under the same conditions in the absence of the antibody. The antibody may selectively and/or significantly reduce or inhibit the binding of GDF15 to its endogenous receptor. As used herein, the term “significantly reduces or inhibits binding” of GDF15 to its receptor is understood to mean that the antibody inhibits GDF15 binding with a potency or percent inhibition that measures at least 10%, preferably 20%, 30% or 40%, and more preferably at least about 50%, 60%, 80% or 90% of GDF15 (serum level/activity) in the absence of said antibody. Binding can be measured using a direct or sandwich enzyme-linked immunosorbent assay (ELISA), as described, e.g., in Tsai et al., 2013, PLOS ONE, 8:e55174. As used herein, the term “selectively” in the context of an antibody that binds to GDF15 or GDF15 receptor is understood to mean that the antibody binds GDF15 or a GDF15 receptor with a binding affinity that is at least two, three, four, five or ten times greater than that of a functionally unrelated protein or another member of the TGF-β superfamily or a receptor of a member of the TGF-superfamily.

Methods for producing antibodies, e.g., those disclosed herein, are known in the art. For example, DNA molecules encoding light chain variable regions and/or heavy chain variable regions can be synthesized chemically or by recombinant DNA methodologies. For example, the sequences of the antibodies can be cloned from hybridomas by conventional hybridization techniques or polymerase chain reaction (PCR) techniques, using the appropriate synthetic nucleic acid primers. The resulting DNA molecules encoding the variable regions of interest can be ligated to other appropriate nucleotide sequences, including, for example, constant region coding sequences, and expression control sequences, to produce conventional gene expression constructs (i.e., expression vectors) encoding the desired antibodies. Production of defined gene constructs is within routine skill in the art.

Nucleic acids encoding desired antibodies can be incorporated (ligated) into expression vectors, which can be introduced into host cells through conventional transfection or transformation techniques. Exemplary host cells are E. coli cells, P. pastoris cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), PER.C6 cells, and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments, the expression vector optionally contains sequences encoding all or part of a constant region, enabling an entire, or a part of, a heavy or light chain to be expressed. The gene construct can be introduced into eukaryotic host cells using conventional techniques.

In certain embodiments, the host cells express an antibody comprising V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing (i) a polypeptide comprising an entire, or part of, a heavy chain or heavy chain variable region, and/or (ii) a polypeptide comprising an entire, or part of, a light chain or light chain variable region. In some embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region, optionally comprising a sialidase fused thereto).

A polypeptide comprising, e.g., an antibody comprising an immunoglobulin heavy chain variable region and/or light chain variable region, can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

In certain embodiments, an antibody can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse

immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. Nos. 6,893,625 (Robinson); 5,500,362 (Robinson); and 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an anti-GDF15 antibody are grafted into human FRs or consensus human FRs. To create consensus human FRs, FRs from several human heavy chain or light chain amino acid sequences are aligned to identify a consensus amino acid sequence. CDR grafting is described in U.S. Pat. Nos. 7,022,500 (Queen); 6,982,321 (Winter); 6,180,370 (Queen); 6,054,297 (Carter); 5,693,762 (Queen); 5,859,205 (Adair); 5,693,761 (Queen); 5,565,332 (Hoogenboom); 5,585,089 (Queen); 5,530,101 (Queen); Jones et al., 1986, NATURE 321: 522-525; Riechmann et al., 1988, NATURE 332: 323-327; Verhoeyen et al., 1988, SCIENCE 239: 1534-1536; and Winter, 1998, FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169: 1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains are said to be produced. See, e.g., U.S. Pat. Nos. 6,706,477 (Zauderer); 6,800,442 (Zauderer); and 6,872,518 (Zauderer).

Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection.

Another approach for modifying a mouse antibody into a form suitable for medical use in humans is HUMAN ENGINEERING™ technology, which is practiced commercially by XOMA (US) LLC. See, e.g., PCT Publication No. WO 93/11794 and U.S. Pat. Nos. 5,766,886 (Studnicka); 5,770,196 (Studnicka); 5,821,123 (Studnicka); and 5,869,619 (Studnicka).

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human mAbs can also be prepared and optimized from phage display libraries by techniques referenced in, e.g., Knappik et al., J. MOL. BIOL. 296:57-86, 2000; and Krebs et al., J. IMMUNOL. METH. 254:67-84 2001).

It is contemplated that variants and derivatives of GDF15 that act as decoys can be useful in the practice of the invention. For example, through deletion analysis, it may be possible to identify smaller biologically active fragments of GDF15 that compete with endogenous GDF15 for its cognate receptor. Similarly, it is possible to create soluble biologically active fragments of the GDF15 receptor that compete with endogenous GDF15 receptor for available GDF. For example, “biologically active fragments” include, but are not limited to, fragments of a naturally-occurring GDF15 (or homolog) or a GDF15 receptor (or homolog) that compete with endogenous GDF15 or an endogenous GDF15 receptor, respectively, for binding to a cognate binding partner (e.g., GDF15 receptor or GDF15, respectively). Exemplary GDF15 ligand traps are described in Yung et al. (2014) Circulation 130:A17285, and U.S. Patent Application Publication No. 2019/036587.

It is contemplated that antisense nucleic acids (DNA and RNA) and small interfering nucleic acids (e.g., siRNAs) can be designed and used using techniques known in the art. Exemplary siRNA inhibitors of GDF15 include siRNAs from Santa Cruz Biotech (Catalog No. sc-39799, targeting mouse GDF15; and Catalog No. sc-39798, targeting human GDF15), siRNAs from Life Technologies (Cat. Nos. AM16708, 4392420, and 1299001, targeting human GDF15; and Cat. Nos. 1320001 and 4390771, targeting mouse GDF15; and Cat. Nos. 1330001 and 4390771, targeting rat GDF15), siRNAs from Fisher Scientific (Catalog No. NC0683807, targeting human GDF15), siRNAs from Origene (Catalog No. SR306321, targeting human GDF15), siRNAs from amsbio (Catalog No. SR509800, targeting rate GDF15), siRNAs from Dharmacon (including Catalog No. D-019875-02, targeting human GDF15), siRNAs from Sigma-Aldrich (Catalog No. EHU052901, targeting human GDF15), and siRNAs described in Kim et al., 2005, MOLECULAR CANCER THERAPEUTICS, 4:487-493, Chang et al., 2007, MOL. CANCER THERAPEUTICS, 6:2271-2279, and Boyle et al., 2009, J. INVEST. DERMATOL., 129:383-391.

Additional exemplary GDF15 modulators include small molecule inhibitors and polypeptide inhibitors, such as those described in Herbertz et al. (2015) Drug Des Devel Ther. 9:4479-4499, and U.S. Patent Application Publication No. 20180282403.

### Therapeutic Uses

The compositions and methods disclosed herein can be used to treat various forms of ocular disorders in a subject. The invention provides methods for reducing ocular fibrosis and/or treating an ocular fibrosis disorder in a subject. The methods comprise administering to the subject an effective amount of a GDF15 modulator, e.g., an anti-GDF15 antibody, either alone or in a combination with another therapeutic agent, to reduce ocular fibrosis or treat the disorder in the subject.

The term “effective amount” as used herein refers to the amount of an active agent (e.g., a GDF15 modulator, e.g., an anti-GDF15 antibody) sufficient to effect beneficial or desired results. An effective amount can be administered in one or more administrations, applications or dosages and is not intended to be limited to a particular formulation or administration route.

As used herein, the term “ocular fibrosis disorder” refers to a disorder that is mediated, enhanced or otherwise facilitated by or associated with fibrosis in an ocular tissue. In the present invention, “ocular tissue” means any tissue present in the eye. In the present invention, ocular tissue is particularly cells constituting the retina or layers constituting the retina. Examples of cells constituting the retina include retinal ganglion cells, amacrine cells, horizontal cells, Muller glial cells, bipolar cells, retinal photoreceptor cells (cones and rods), retinal pigment epithelial cells, and the like. Examples of layers constituting the retina include the internal limiting membrane, the nerve fiber layer, the ganglionic cell layer, the internal reticular membrane, the internal nuclear layer, the external plexiform layer, the external nuclear layer, the external limiting membrane, the photoreceptor cell layer, and the retinal pigment epithelial layer.

In the present invention, among these, the ocular tissue is preferably retinal pigment epithelial cells.

Examples of ocular fibrosis disorders include wet age-related macular degeneration, intractable retinal vitreous diseases such as proliferative vitreoretinopathy and diabetic retinopathy, diabetic macular edema (DME), retinal hemorrhage and retinal detachment. Further examples of ocular disorders include those associated with macular degeneration, such as presbyopia (an age-related form of farsightedness), choroidal neovascularization, subfoveal or juxtafoveal neovascularization, corneal astigmatism and lenticular astigmatism (See U.S. Pat. No. 9,226,940, and U.S. Patent Application Publication No. 20170326106, both of which are hereby incorporated herein by reference).

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

The ocular tissue fibrosis inhibitor of the present invention is effective for animals, including humans, and is especially effective for mammals, including humans. Note, when the ocular tissue fibrosis inhibitor of the present invention is administered to a human, the anti-GDF15 antibody may be modified to be capable of being administered to humans by a known method as needed insofar as the effect of the present invention is not impaired.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a GDF15 modulator is administered in combination with an anti-angiogenic agent. In certain embodiments, the anti-angiogenic agent is selected from aflibercept, an anti-VEGF antibody (e.g., bevacizumab and ranibizumab), sunitinib, pazopanib, sorafenib, regorafenib, vandetanib, cabozantinib, axitinib, tivozanib, linifanib. pegaptanib, spironolactone, indomethacin, thalidomide, interleukin-12, an anti-FGF antibody, a tyrosine kinase inhibitor, an interferon, suramin, a suramin analog, somatostatin, and a somatostatin analog. In certain embodiments, the anti-angiogenic agent is a VEGF inhibitor, e.g., a VEGF inhibitor selected from aflibercept, bevacizumab, ranibizumab, sunitinib, pazopanib, sorafenib, regorafenib, vandetanib, cabozantinib, axitinib, tivozanib and linifanib. In certain embodiments, the anti-angiogenic agent is bevacizumab.

### Formulation and Delivery

Pharmaceutical compositions containing GDF15 modulators, such as those disclosed herein, can be formulated into dosage forms or dosage units using standard formulation techniques. However, the pharmaceutical composition should be formulated to be compatible with its intended route of administration. As needed, the ocular tissue fibrosis inhibitor of the present invention may have pharmaceutically acceptable additives thereto and may be formulated as single preparations or compound preparations using commonly used art.

The compositions described herein can be administered to a subject via any route, including, but not limited to, intravenous (e.g., by infusion pumps), intraperitoneal, intraocular, intra-arterial, intrapulmonary, oral, inhalation, intravesicular, intramuscular, intra-tracheal, subcutaneous, intraocular, intrathecal, transdermal, transpleural, intraarterial, topical, inhalational (e.g., as mists of sprays), mucosal (such as via nasal mucosa), subcutaneous, transdermal, gastrointestinal, intraarticular, intracistemal, intraventricular, rectal (i.e., via suppository), vaginal (i.e., via pessary), intracranial, intraurethral, intrahepatic, and intratumoral. In some embodiments, the compositions are administered systemically (for example by intravenous injection). In some embodiments, the compositions are administered locally (for example by intraarterial or intraocular injection). In some embodiments, the compositions are administered either parenterally or orally. Examples of dosage forms for parenteral administration include eye drops, injections, nasal drops, and the like, examples of dosage forms for oral administration include tablets, capsules, fine granules, pellets, and the like, and these may be formulated using commonly used art. Note, when an injection is used, this may be administered by ocular, intraocular, intravitreal, subconjunctival, intravenous, intraarterial, subdermal, intradermal, or intramuscular injection or the like.

Useful formulations can be prepared by methods well known in the pharmaceutical art. For example, see REMINGTON'S PHARMACEUTICAL SCIENCES, 18th ed. (Mack Publishing Company, 1990). Formulation components suitable for parenteral administration include a sterile diluent such as bacteriostatic water for injection, physiological saline, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose. The carrier should be stable under the conditions of manufacture and storage, and should be preserved against microorganisms. In some embodiments, the composition (e.g., an antibody) is lyophilized, and then reconstituted in buffered saline, at the time of administration.

For therapeutic use, the composition (e.g., an antibody) preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

For example, eye drops may be suitably compounded with isotonic agents, buffers, pH adjusters, solubilizers, thickeners, stabilizers, preservatives, and the like. Furthermore, stable eye drops may be obtained by suspending the drug by adding pH regulators, thickeners, dispersants, and the like. Examples of isotonic agents include glycerol, propylene glycol, sodium chloride, potassium chloride, sorbitol, mannitol, and the like Examples of buffers include phosphoric acid, phosphate, citric acid, acetic acid, ϵ-aminocaproic acid, and the like. Examples of pH regulators include hydrochloric acid, citric acid, phosphoric acid, acetic acid, sodium hydroxide, potassium hydroxide, boric acid, borax, sodium carbonate, sodium bicarbonate, and the like. Examples of solubilizing agents include polysorbate 80, polyoxyethylene-hydrogenated castor oil 60, macrogol 4000, and the like. Examples of thickeners and dispersants include cellulose polymers such as hydroxypropyl methylcellulose and hydroxypropyl cellulose, polyvinyl alcohol, polyvinyl pyrrolidone, and the like, and examples of stabilizers include edetate, sodium edetate, and the like. Examples of preservatives (antiseptic agents), include general-purpose sorbic acid, potassium sorbate, benzalkonium chloride, benzethonium chloride, methyl parahydroxybenzoate, propyl parahydroxybenzoate, chlorobutanol, and the like.

The pH of the eye drops may be any within the range allowed for eye drops, but it is preferably in the range of 4.0 to 8.5, and the osmotic pressure ratio is preferably set to around 1.0.

The pharmaceutical compositions preferably are sterile. Sterilization can be accomplished, for example, by filtration through sterile filtration membranes. Where the composition is lyophilized, filter sterilization can be conducted prior to or following lyophilization and reconstitution.

Tablets may be formulated by suitably selecting for use, for example: excipients such as lactose, glucose, D-mannitol, anhydrous calcium hydrogen phosphate, starch, and sucrose; disintegrants such as carboxymethylcellulose, carboxymethyl cellulose calcium, croscarmellose sodium, crospovidone, starch, partially pregelatinized starch, and low-substituted hydroxypropyl cellulose; binders such as hydroxypropyl cellulose, ethylcellulose, gum arabic, starch, partially pregelatinized starch, polyvinylpyrrolidone, and polyvinyl alcohol; lubricants such as magnesium stearate, calcium stearate, talc, hydrous silicon dioxide, and hydrogenated oil; coating agents such as purified sucrose, hydroxypropyl methylcellulose, hydroxypropyl cellulose, methylcellulose, ethylcellulose, and polyvinylpyrrolidone; flavoring agents such as citric acid, aspartame, ascorbic acid, and menthol; and the like.

Furthermore, injections include solutions, suspensions, emulsions and solid injections to be used by dissolving or suspending in a solution at the time of use, and for example, the ocular tissue fibrosis inhibitor of the present invention is dissolved, suspended, or emulsified in a solution. At this time, the ocular tissue fibrosis inhibitor of the present invention may be subjected to treatment such as freeze-drying. The injection may be formulated by suitably selecting for use, for example: diluents such as bacteriostatic water for injection, physiological saline, oil, polyethylene glycol, glycerin, and propylene glycol; antibacterial agents such as benzyl alcohol and methylparaben; antioxidants such as ascorbic acid and sodium sulfite; chelating agents such as EDTA; buffer solutions such as acetate, citrate, and phosphoric acid; isotonic agents such as sodium chloride and dextrose; and the like.

Generally, a therapeutically effective amount of active component is in the range of 0.1 mg/kg to 100 mg/kg, e.g., 1 mg/kg to 100 mg/kg, 1 mg/kg to 10 mg/kg. A therapeutically effective amount of active component may also be in the range of 1 mg/m² to 1000 mg/m², e.g., 1 mg/m² to 500 mg/m² or 50 mg/m² to 500 mg/m² or 200 mg/m² to 700 mg/m² or 300 mg/m² to 600 mg/m² or 400 mg/m² to 800 mg/m² or 500 mg/m² to 1000 mg/m². The amount administered will depend on variables such as the type and extent of disease or indication to be treated, the overall health of the patient, the in vivo potency of the composition (e.g., an antibody), the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the composition (e.g., an antibody), and the disease being treated. Exemplary dosing frequencies are once per day, once per week and once every two weeks.

The optimal effective amount of the compositions can be determined empirically and will depend on the type and severity of the disease, route of administration, disease progression and health, mass and body area of the subject. Such determinations are within the skill of one in the art. Examples of dosages of GDF15 modulator molecules which can be used for methods described herein include, but are not limited to, an effective amount within the dosage range of any of about 0.01 μg/kg to about 300 mg/kg, or within about 0.1 μg/kg to about 40 mg/kg, or with about 1 μg/kg to about 20 mg/kg, or within about 1 μg/kg to about 10 mg/kg. For example, when administered subcutaneously, the composition may be administered at low microgram ranges, including for example about 0.1 μg/kg or less, about 0.05 μg/kg or less, or 0.01 μg/kg or less.

In certain embodiments, the amount of GDF15 modulators administered to a subject is about 10 μg to about 500 mg per dose, including for example any of about 10 μg to about 50 μg, about 50 μg to about 100 μg, about 100 μg to about 200 μg, about 200 μg to about 300 μg, about 300 μg to about 500 μg, about 500 μg to about 1 mg, about 1 mg to about 10 mg, about 10 mg to about 50 mg, about 50 mg to about 100 mg, about 100 mg to about 200 mg, about 200 mg to about 300 mg, about 300 mg to about 400 mg, or about 400 mg to about 500 mg per dose. In certain embodiments, a GDF15 modulator is administered at a dose from about 0.025 mg to about 4 mg, from about 0.035 mg to about 2 mg, from about 0.05 mg to about 2 mg, from about 0.1 mg to about 2 mg, from about 0.2 mg to about 1 mg, or from about 0.2 mg to about 0.8 mg of the GDF15 modulator can be administered. In one embodiment, 0.5 mg of GDF15 modulator is administered locally. In certain other embodiments, from about 0.05 mg to about 2 mg, from about 0.2 mg to about 2 mg, from about 0.05 mg to about 1.5 mg, from about 0.15 mg to about 1.5 mg, from about 0.4 mg to about 1 mg, or from about 0.5 mg to about 0.8 mg of GDF15 modulator is administered locally.

The GDF15 modulator compositions may be administered in a single daily dose, or the total daily dose may be administered in divided dosages of two, three, or four times daily. The compositions can also be administered less frequently than daily, for example, six times a week, five times a week, four times a week, three times a week, twice a week, once a week, once every two weeks, once every three weeks, once a month, once every two months, once every three months, or once every six months. The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection or surgical implanted in various locations in the body.

In certain embodiments of the invention, the dosing of the GDF15 modulator is titrated such that the dose is sufficient to reduce or prevent adverse effects, but yet fully or partially inhibit the activity of the GDF15.

In certain embodiments, a 0.001 to 10% (w/v) dose is administered once to multiple times a day for eye drops. In certain embodiments, 0.1 mg/kg to 100 mg/kg per day is administered in one dose or multiple doses for injections. Furthermore, 0.1 mg/kg to 100 mg/kg of an oral agent per day may usually be administered in one dose or multiple doses. Moreover, amounts outside the foregoing range may be used as needed.

In some aspects, the activity of GDF15 can be modulated in a target cell using antisense nucleic acids or small interfering nucleic acids. Modulation can be achieved using expression constructs known in the art, e.g., naked DNA constructs, DNA vector based constructs, and/or viral vector and/or viral based constructs to express nucleic acids encoding an anti-GDF15 siRNA or antisense molecule.

Exemplary DNA constructs and the therapeutic use of such constructs are well known to those of skill in the art (see, e.g., Chiarella et al., 2008, RECENT PATENTS ANTI-INFECT. DRUG DISC., 3:93-101; Gray et al., 2008, EXPERT OPIN. BIOL. THER., 8:911-922; Melman et al., 2008, HUM. GENE THER., 17:1165-1176). Naked DNA constructs typically include one or more therapeutic nucleic acids (e.g., GDF15 modulators) and a promoter sequence. A naked DNA construct can be a DNA vector, commonly referred to as pDNA. Naked DNA typically do not integrate into chromosomal DNA. Generally, naked DNA constructs do not require, or are not used in conjunction with, the presence of lipids, polymers, or viral proteins. Such constructs may also include one or more of the non-therapeutic components described herein.

DNA vectors are known in the art and typically are circular double stranded DNA molecules. DNA vectors usually range in size from three to five kilo-base pairs (e.g., including inserted therapeutic nucleic acids). Like naked DNA, DNA vectors can be used to deliver and express one or more therapeutic proteins in target cells. DNA vectors do not integrate into chromosomal DNA.

Generally, DNA vectors include at least one promoter sequence that allows for replication in a target cell. Uptake of a DNA vector may be facilitated by combining the DNA vector with, for example, a cationic lipid, and forming a DNA complex. Typically, viral vectors are double stranded circular DNA molecules that are derived from a virus. Viral vectors typically are larger in size than naked DNA and DNA vector constructs and have a greater capacity for the introduction of foreign (i.e., not virally encoded) genes. Like naked DNA and DNA vectors, viral vectors can be used to deliver and express one or more therapeutic nucleic acids in target cells. Unlike naked DNA and DNA vectors, certain viral vectors stably incorporate themselves into chromosomal DNA. Typically, viral vectors include at least one promoter sequence that allows for replication of one or more vector encoded nucleic acids, e.g., a therapeutic nucleic acid, in a host cell. Viral vectors may optionally include one or more non-therapeutic components described herein. Advantageously, uptake of a viral vector into a target cell does not require additional components, e.g., cationic lipids. Rather, viral vectors transfect or infect cells directly upon contact with a target cell.

The approaches described herein include the use of retroviral vectors, adenovirus-derived vectors, and/or adeno-associated viral vectors as recombinant gene delivery systems for the transfer of exogenous genes in vivo, particularly into humans. Protocols for producing recombinant retroviruses and for infecting cells in vitro or in vivo with such viruses can be found in CURRENT PROTOCOLS IN MOLECULAR BIOLOGY, Ausubel, F. M. et al. (eds.) Greene Publishing Associates, (1989), Sections 9.10-9.14, and other standard laboratory manuals.

Viruses that are used as transduction agents of DNA vectors and viral vectors such as adenoviruses, retroviruses, and lentiviruses may be used in practicing the present invention. Illustrative retroviruses include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus. As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In certain embodiments, an adenovirus can be used in accordance with the methods described herein. The genome of an adenovirus can be manipulated such that it encodes and expresses a gene product of interest but is inactivated in terms of its ability to replicate in a normal lytic viral life cycle. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors.

Adeno-associated virus is a naturally occurring defective virus that requires another virus, such as an adenovirus or a herpes virus, as a helper virus for efficient replication and a productive life cycle. It is also one of the few viruses that may integrate its DNA into non-dividing cells, and exhibits a high frequency of stable integration.

In various embodiments, one or more viral vectors that expresses a therapeutic transgene or transgenes encoding a GDF15 modulator is administered by direct injection to a cell, tissue, or organ of a subject, in vivo. In various other embodiments, cells are transduced in vitro or ex vivo with such a vector encapsulated in a virus, and optionally expanded ex vivo. The transduced cells are then administered to the subject. Cells suitable for transduction include, but are not limited to stem cells, progenitor cells, and differentiated cells. In certain embodiments, the transduced cells are embryonic stem cells, bone marrow stem cells, umbilical cord stem cells, placental stem cells, mesenchymal stem cells, neural stem cells, liver stem cells, pancreatic stem cells, cardiac stem cells, kidney stem cells, or hematopoietic stem cells.

In particular embodiments, host cells transduced with viral vector of the invention that expresses one or more polypeptides, are administered to a subject to treat and/or prevent an auditory disease, disorder, or condition. Other methods relating to the use of viral vectors, which may be utilized according to certain embodiments of the present invention, can be found in, e.g., Kay, 1997, CHEST, 111 (6 Supp.): 138S-142S; Ferry et al., 1998, HUM. GENE THER., 9:1975-81; Shiratory et al., 1999, LIVER, 19:265-74; Oka et al., 2000, CURR. OPIN. LIPIDOL., 11:179-86; Thule et al., 2000, GENE THER., 7: 1744-52; Yang, 1992, CRIT. REV. BIOTECHNOL., 12:335-56; Alt, 1995, J. HEPATOL., 23:746-58; Brody et al., 1994, ANN. N.Y. ACAD. SCI., 716:90-101; Strayer, 1999, EXPERT OPIN. INVESTIG. DRUGS, 8:2159-2172; Smith-Arica et al., 2001, CURR. CARDIOL. REP., 3:43-49; and Lee et al., 2000, NATURE, 408:483-8.

Certain embodiments of the invention provide conditional expression of a polynucleotide of interest. For example, expression is controlled by subjecting a cell, tissue, organism, etc., to a treatment or condition that causes the polynucleotide to be expressed or that causes an increase or decrease in expression of the polynucleotide encoded by the polynucleotide of interest. Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, GENE, 323:67), the cumate inducible gene switch (WO 2002/088346), tetracycline-dependent regulatory systems, etc.

Conditional expression can also be achieved by using a site specific DNA recombinase. According to certain embodiments of the invention the vector comprises at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, 1993, CURRENT OPINION IN BIOTECHNOLOGY, 3:699-707), or mutants, derivatives (e.g., fusion proteins containing the recombination protein sequences or fragments thereof), fragments, and variants thereof. Illustrative examples of recombinases suitable for use in particular embodiments of the present invention include, but are not limited to: Cre, Int, IHF, Xis, Flp, Fis, Hin, Gin, OC31 , Cin, Tn3 resolvase, TndX, XerC, XerD, TnpX, Hjc, Gin, SpCCEl. and ParA.

The vectors may comprise one or more recombination sites for any of a wide variety of site specific recombinases. It is to be understood that the target site for a site specific recombinase is in addition to any site(s) required for integration of a vector (e.g., a retroviral vector or lentiviral vector).

In certain embodiments, vectors comprise a selection gene, also termed a selectable marker. Typical selection genes encode proteins that (a) confer resistance to antibiotics or other toxins, e.g., ampicillin, neomycin, hygromycin, methotrexate, Zeocin, Blastocidin, or tetracycline, (b) complement auxotrophic deficiencies, or (c) supply critical nutrients not available from complex media, e.g., the gene encoding D-alanine racemase for Bacilli. Any number of selection systems may be used to recover transformed cell lines. These include, but are not limited to, the herpes simplex virus thymidine kinase (Wigler et al., 1977, CELL, 11:223-232) and adenine phosphoribosyltransferase (Lowy et al., 1990, CELL, 22:817-823) genes which can be employed in tk- or aprt-cells, respectively.

All the molecular biological techniques required to generate an expression construct described herein are standard techniques that will be appreciated by one of skill in the art.

In certain embodiments, DNA delivery may occur parenterally, intravenously, intramuscularly, or even intraperitoneally as described, for example, in U.S. Pat. Nos. 5,543,158; 5,641,515; and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

In certain embodiments, DNA delivery may occur by use of liposomes, nanocapsules, microparticles, microspheres, lipid particles, vesicles, optionally mixing with cell penetrating polypeptides, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

Exemplary formulations for ex vivo DNA delivery may also include the use of various transfection agents known in the art, such as calcium phosphate, electroporation, heat shock and various liposome formulations (i.e., lipid-mediated transfection). Particular embodiments of the invention may comprise other formulations, such as those that are well known in the pharmaceutical art, and are described, for example, in REMINGTON: THE SCIENCE AND PRACTICE OF PHARMACY, 20th Edition. Baltimore, MD: Lippincott Williams & Wilkins, 2000.

In certain embodiments, GDF15 activity is inhibited by contacting a body fluid with a composition comprising a GDF15 modulator ex vivo under conditions that permit the GDF15 modulators to reduce or inhibit GDF15 activity. Suitable body fluids include those that can be returned to the individual, such as blood, plasma, or lymph. Affinity adsorption apheresis is described generally in Nilsson et al., 1988, BLOOD, 58 (1):38-44; Christie et al., 1993, TRANSFUSION, 33:234-242; Richter et al., 1997, ASAIO J., 43 (1):53-59; Suzuki et al., 1994, AUTOIMMUNITY, 19: 105-112; U.S. Pat. No. 5,733,254; Richter et al., 1993, METABOL. CLIN. EXP., 42:888-894; and Wallukat et al., 1996, INT'L, J. CARD., 54:1910195.

Accordingly, the invention includes methods of treating one or more diseases described herein in a subject comprising treating the subject's blood extracoporeally (i.e., outside the body or ex vivo) with a composition comprising a GDF15 modulator under conditions that permit the modulator to reduce or inhibit GDF15 activity in the blood of the subject.

The ocular tissue fibrosis inhibitor of the present invention may be used together with other known active ingredients (for example, anticancer agents and the like) for the purpose of combination treatment or the like. Furthermore, the ocular tissue fibrosis inhibitor of the present invention may be used in combination with a known sustained-release device or DDS device such as microspheres, nanospheres, nanoparticles, liposomes, or micelles.

The ocular tissue fibrosis inhibitor of the present invention is also useful as an oral or enteric nutrient supplement (food or drink). Various conventionally known forms and kinds of food and drink may be adopted. Furthermore, the ocular tissue fibrosis inhibitor of the present invention may be used as a food additive. When ingested as a nutritional supplement, about 0.1 mg/kg to 100 mg/kg per day is ingested in one or multiple doses. Moreover, amounts outside the foregoing range may be used as needed.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a +10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The present invention will be described in further detail below based on examples.

Note, the examples described below show one example of representative examples of the present invention, and the scope of the present invention is not to be narrowly interpreted due to such.

### Example 1

In the present experimental example 1, the localization and expression variation of GDF15 in a laser-induced choroidal neovascularization model (choroidal neovascularization: CNV, also referred to as “CNV”) were examined.

A 7:1 mixture anesthetic solution of ketamine and xylazine was diluted 10 times (1 mL/kg) using physiological saline and administered into the femoral muscle of mice. Then, 0.1% Hyalein (registered trademark) eye drops were administered such that eyes did not dry out. While fixing a cover glass to the right eye, the ocular fundus was viewed, and a laser coagulator (MC500) was used to irradiate six locations around the circumference of the optic disk with laser beams at equal intervals (wavelength: 647 nm, spot size: 50 μm, time: 100 msec, laser output: 120 mW).

Sampling was conducted 3, 14, and 28 days after laser irradiation, and change in expression of GDF15 and type I collagen (fibrosis marker) was examined by immunostaining. In addition, the retinal pigment epithelium and the choroid complex were sampled 1, 3, 5, 7, and 14 days after laser irradiation, and change in expression of GDF15 was examined by western blotting.

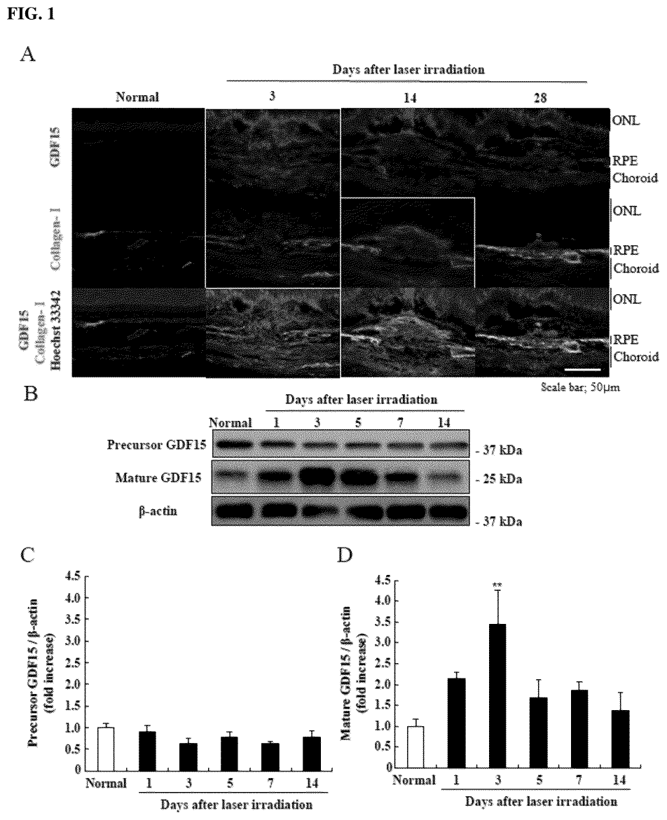

FIG. 1A illustrates localization and variation in expression of GDF15. An increase in the level of expression of GDF15 was observed at sites of angiogenesis 3, 14, and 28 days after laser irradiation. Furthermore, expression of GDF15 was observed near type I collagen accumulation sites 14 days after laser irradiation.

FIGS. 1B, C and D illustrate variation in expression of activated GDF15. The level of expression of activated GDF15 increased in the retinal pigment epithelium to choroid.

Accordingly, it was suggested that GDF15 is involved in fibrous scarring formation associated with choroid neovascularization.

### Example 2

In the present experimental example 2, sources of production of GDF15, which is expressed at high levels in CNV lesions, were examined.

A 7:1 mixture anesthetic solution of ketamine and xylazine was diluted 10 times (1 mL/kg) using physiological saline and administered into the femoral muscle of mice. Then, 0.1% Hyalein (registered trademark) eye drops were administered such that eyes did not dry out. While fixing a cover glass to the right eye, the ocular fundus was viewed, and a laser coagulator (MC500) was used to irradiate six locations around the circumference of the optic disk with laser beams at equal intervals (wavelength: 647 nm, spot size: 50 μm, time: 100 msec, laser output: 120 mW).

Sampling was conducted 3 days after laser irradiation, and change in expression of GDF15 and Iba-1 (macrophage marker) was examined by immunostaining.

RAW264.7, a macrophage cell, was seeded in a 12 well plate at 1.5×10⁶ cells/well and cultured for 1 day. The culture medium was replaced under 1% FBS conditions and cultured for 1 hour. Then, lipopolysaccharide (LPS), an inflammation-inducing agent, was added. Sampling was performed 24 hours after addition, and change in protein expression of GDF15 was examined by western blotting.

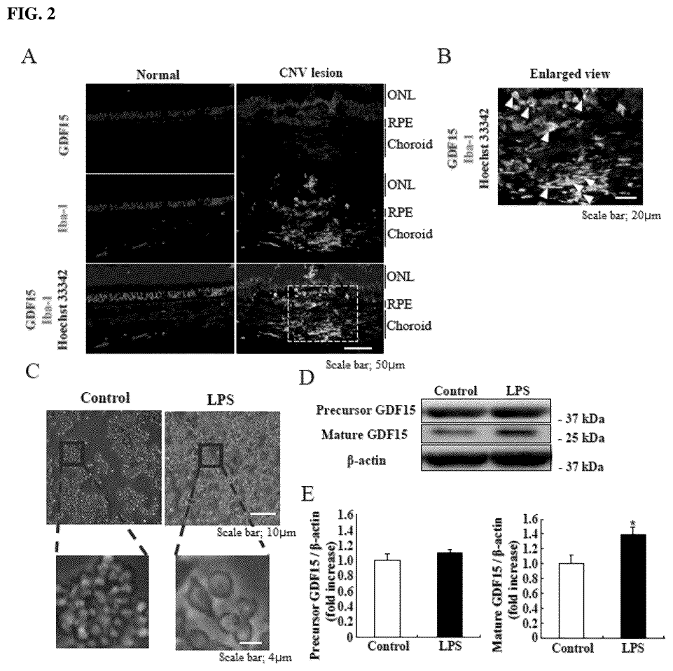

FIGS. 2A-B illustrate localization of GDF15 and macrophage markers. In the CNV lesion, GDF15 was co-localized with Iba-1 (macrophage marker).

FIGS. 2C, D, and E illustrate the expression of GDF15 in activated macrophage cells. LPS treatment increased the production of activated GDF15 in macrophage cells.

Accordingly, it was suggested that the source of the production of GDF15, expression of which is elevated under fibrosis pathology conditions, is macrophages.

### Example 3

In the present experimental example 3, the effect of GDF15 treatment on cell morphology of retinal pigment epithelial cells was examined.

ARPE-19, a human retinal pigment epithelial cell strain, was seeded in a 24 well plate at 2.5×10⁴ cells/well and cultured for 4 days until 90% confluent. Upon reaching 90%, the culture medium was replaced under 1% FBS conditions and cultured for 24 hours. After replacing the culture medium again, human recombinant GDF15 and human recombinant TGFβ1, which is known as a profibrotic factor, were added. The drug was added every 2 days, and morphological changes were observed 144 hours after addition.

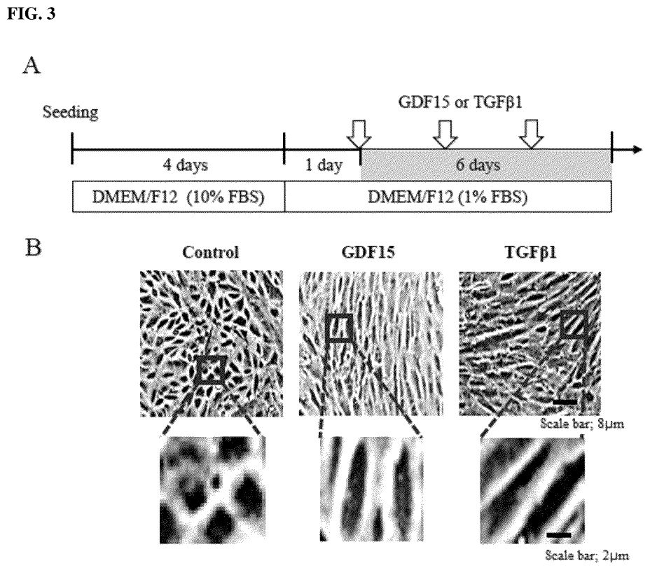

FIGS. 3A-B illustrate the effect of GDF15 treatment on cell morphology of retinal pigment epithelial cells. The addition of GDF15 induced EMT-like morphological changes in which the cell morphology becomes spindle-shaped.

Accordingly, it was suggested that GDF15 promotes fibrotic transformation of retinal pigment epithelial cells.

### Example 4

In the present experimental example 4, the effect of GDF15 treatment on extracellular matrix production capacity of retinal pigment epithelial cells was examined.

ARPE-19, a human retinal pigment epithelial cell strain, was seeded in 24 and 96 well plates at 2.5×10⁴ cells/well and 5.0×10³ cells/well, respectively, and cultured for 4 days until 90% confluent. Upon reaching 90%, the culture medium was replaced under 1% FBS conditions and cultured for 24 hours. After replacing the culture medium again, human recombinant GDF15 (AVISCERA BIOSCIENCE) and human recombinant TGFβ1 (R&D Systems), which is known as a fibrosis promoting factor, were added. The drug was added every 2 days, sampling was carried out 144 hours after addition, and change in protein expression of fibronectin (fibrosis marker) was examined by western blotting and immunostaining.

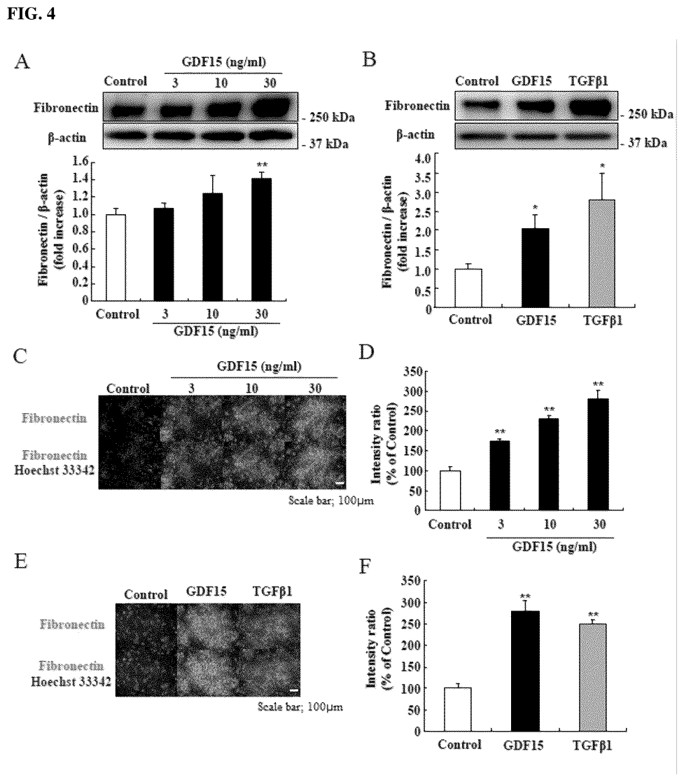

FIGS. 4A-F illustrate variation in protein expression of fibronectin due to the addition of GDF15. The expression of fibronectin, a fibrosis marker, increased due to the addition of GDF15.

Accordingly, it was suggested that GDF15 acts on retinal pigment epithelial cells to promote fibrosis.

### Example 5

In the present experimental example 5, the action of neutralizing antibodies targeting GDF15 was examined.

ARPE-19, a human retinal pigment epithelial cell strain, was seeded in a 96 well plate at 5.0×10³ cells/well and cultured for 4 days until 90% confluent. Upon reaching 90%, the culture medium was replaced under 1% FBS conditions and cultured for 24 hours. After replacing the culture medium again, human GDF-15 antibody (product number: AF957, made by R&D, source: polyclonal goat IgG) was added, and human recombinant GDF15 (AVISCERA BIOSCIENCE) was added 30 minutes later. The drug was added every 2 days, sampling was carried out 144 hours after addition, and change in protein expression of fibronectin (fibrosis marker) was examined by immunostaining.

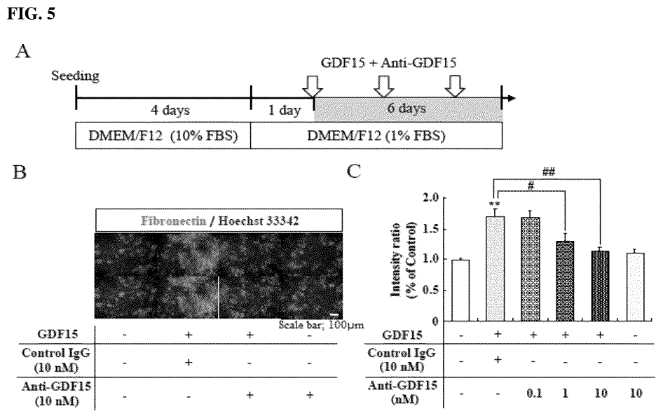

FIGS. 5A-B illustrate the effect of anti-GDF15 antibodies on fibrosis of retinal pigment epithelial cells. Increase in GDF 15-induced fibronectin expression was suppressed in a concentration-dependent manner by addition of anti-GDF15 antibodies.

Accordingly, it is suggested that the addition of anti-GDF15 antibodies is useful for suppressing fibrosis of retinal pigment epithelial cells.

### Example 6

In the present experimental example 6, the mechanism of GDF15 induction promoting the fibrosis was examined.

ARPE-19, a human retinal pigment epithelial cell strain, was seeded in a 48 well plate at 1.25×10⁴ cells/well and cultured for 4 days until 90% confluent. Upon reaching 90%, the culture medium was replaced under 0% FBS conditions and cultured for 24 hours. Then, human recombinant GDF15 (AVISCERA BIOSCIENCE) was added. Sampling was performed 15, 30, and 60 minutes after addition, and variation in protein expression of GDF15 downstream signal-related factors was examined by western blotting.

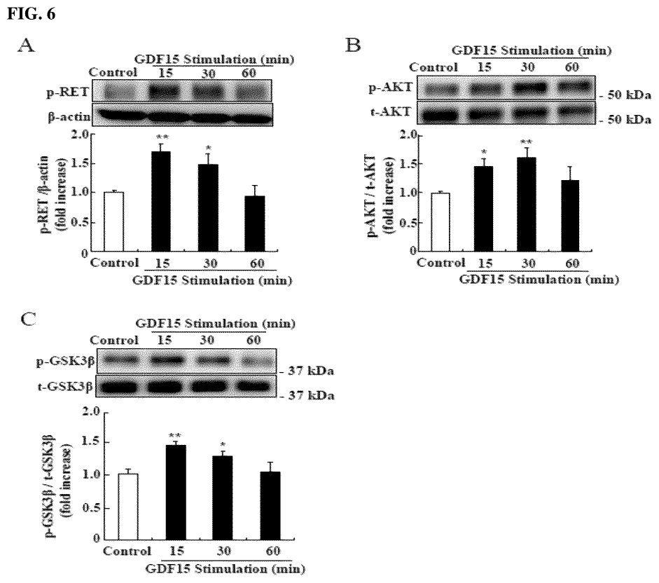

FIGS. 6A-C illustrate variation in expression of downstream signals due to the addition of GDF15. Phosphorylation of rearranged during transfection (RET), AKT, and GSK3β, which are downstream signals, of GFRAL receptor, which is a receptor having high affinity for GDF15, was promoted.

When GDF15 binds to GFRAL, RET receptor approaches and forms a complex between the three, resulting in downstream signals acting. This system is specific to GDF15. Accordingly, it was suggested that GDF 15 binds to GFRAL receptor of retinal pigment epithelial cells, induces phosphorylation of RET, and causes action of downstream signals. Therefore, it was found that GDF15 may contribute to fibrosis of retinal pigment epithelial cells via these mechanisms.

Note, GFRAL receptor is a receptor having high affinity to GDF15 as mentioned above, and thus the foregoing signal does not work for TGFβ and is specific to GDF15.

### Example 7

In the present experimental example 7, the action of neutralizing antibodies targeting GDF15 is examined.

ARPE-19, a human retinal pigment epithelial cell strain, is seeded in a 96 well plate at 5.0×10³ cells/well and cultured for 4 days until 90% confluent. Upon reaching 90%, the culture medium is replaced under 1% FBS conditions and cultured for 24 hours. After replacing the culture medium again, anti-GDF15 antibodies Hu01G06-127 (including SEQ ID NO: 30 and SEQ ID NO: 47) or Hu01G06-135 (including SEQ ID NO: 29 or SEQ ID NO: 48) are added, and human recombinant GDF15 (AVISCERA BIOSCIENCE) is added 30 minutes later. The drug is added every 2 days, sampling is carried out 144 hours after addition, and change in protein expression of fibronectin (fibrosis marker) is examined by immunostaining.

It is expected that an increase in GDF15-induced fibronectin expression is suppressed in a concentration-dependent manner by addition of anti-GDF15 antibodies.

Accordingly, it is expected that anti-GDF15 antibodies, such as Hu01G06-127 and Hu01G06-135, are useful for suppressing fibrosis of retinal pigment epithelial cells and treating ocular-fibrosis disorders such as macular degeneration.

### Example 8

On day 0, a 7:1 mixture anesthetic solution of ketamine and xylazine is diluted 10 times (1 mL/kg) using physiological saline and administered into the femoral muscle of mice. Then, 0.1% Hyalein (registered trademark) eye drops are administered such that eyes do not dry out. While fixing a cover glass to the right eye, the ocular fundus is viewed, and a laser coagulator (MC500) is used to irradiate six locations around the circumference of the optic disk with laser beams at equal intervals (wavelength: 647 nm, spot size: 50 μm, time: 100 msec, laser output: 120 mW).

Mice in the treatment group are administered anti-GDF15 antibody Hu01G06-127 (including SEQ ID NO: 30 and SEQ ID NO: 47) or Hu01G06-135 (including SEQ ID NO: 29 or SEQ ID NO: 48) via intravitreal injection at day 0, 7, 14 and 21 at a concentration of 20 mg/kg. Mice in the control group are administered PBS via intravitreal injection at day 0, 7, 14, and 21. Sampling is conducted 3, 14, and 28 days after laser irradiation, and change in expression of GDF15 and type I collagen (fibrosis marker) is examined by immunostaining. In addition, the retinal pigment epithelium and the choroid complex are sampled 1, 3, 5, 7, and 14 days after laser irradiation, and change in expression of GDF15 is examined by western blotting.

It is expected that mice treated with the anti-GDF15 antibody have reduced GDF15 in the retinal pigment epithelium to choroid and reduced production of fibrosis and scarring as compared to control mice, showing that the anti-GDF15 antibody is an effective treatment to reduce fibrosis in fibrotic ocular disorders.

### INDUSTRIAL APPLICABILITY

According to the present invention, a novel methods of inhibiting ocular tissue fibrosis are provided.

It is suggested that present invention is useful for radical treatment of ocular tissue fibrosis, and it is believed to contribute to the suppression of loss of visual acuity in patients for whom fibrous scarring formation is predicted and lead to rehabilitation of patients and reduction of medical expenses.

### INCORPORATION BY REFERENCE

The entire disclosure of each of the patent documents and scientific articles referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

1. A method of reducing ocular tissue fibrosis in a subject in need thereof, the method comprising administering to the subject an effective amount of a GDF15 modulator thereby to reduce ocular tissue fibrosis in the subject.

2. The method of claim 1, wherein the subject suffers from a disorder selected from macular degeneration, an intractable retinal vitreous disease, diabetic macular edema (DME), retinal hemorrhage, retinal detachment, presbyopia, choroidal neovascularization, subfoveal or juxtafoveal neovascularization, corneal astigmatism, and lenticular astigmatism.

3. A method of treating an ocular fibrosis disorder in a subject in need thereof, the method comprising administering to the subject an effective amount of a GDF15 modulator thereby to treat the disorder in the subject.

4. The method of claim 3, wherein the disorder is selected from macular degeneration, an intractable retinal vitreous disease, diabetic macular edema (DME), retinal hemorrhage, retinal detachment, presbyopia, choroidal neovascularization, subfoveal or juxtafoveal neovascularization, corneal astigmatism, and lenticular astigmatism.

5. The method of claim 2, wherein the macular degeneration is wet age-related macular degeneration.

6. The method of claim 2, wherein the intractable retinal vitreous disease is proliferative vitreoretinopathy or diabetic retinopathy.

7. The method of claim 1, wherein GDF15 modulator decreases or inhibits GDF15 activity.

8. The method of claim 1, wherein the GDF15 modulator is an anti-GDF15 antibody.

9. The method of claim 8, wherein the anti-GDF15 antibody is humanized or human.

10. The method of claim 8, wherein the anti-GDF15 antibody is selected from:
(i) an antibody comprising a CDR_{H1} sequence of SEQ ID NO: 1, a CDR_{H2} sequence of SEQ ID NO:7, and a CDR_{H3} sequence of SEQ ID NO: 13; and a CDR_{L1} sequence of SEQ ID NO: 16, a CDR_{L2} sequence of SEQ ID NO:18, and a CDR_{L3} sequence of SEQ ID NO:22;
(ii) an antibody comprising a CDR_{H1} sequence of SEQ ID NO:1, a CDR_{H2} sequence of SEQ ID NO:9, and a CDR_{H3} sequence of SEQ ID NO: 13; and a CDR_{L1} sequence of SEQ ID NO:16, a sequence of SEQ ID NO: 18, and a CDR_{L3} sequence of SEQ ID NO:22;
(iii) an antibody comprising a CDR_{H1} sequence of SEQ ID NO:1, a CDR_{H2} sequence of SEQ ID NO:4, and a CDR_{H3} sequence of SEQ ID NO: 13; and a CDR_{L1} sequence of SEQ ID NO: 16, a CDR_{L2} sequence of SEQ ID NO: 18, and a CDR_{L3} sequence of SEQ ID NO:21;
(iv) an antibody comprising a CDR_{H1} sequence of SEQ ID NO: 1, a CDR_{H2} sequence of SEQ ID NO:5, and a CDR_{H3} sequence of SEQ ID NO: 13; and a CDR_{L1} sequence of SEQ ID NO: 16, a CDR_{L2} sequence of SEQ ID NO: 18, and a CDR_{L3} sequence of SEQ ID NO:21;
(v) an antibody comprising a CDR_{H1} sequence of SEQ ID NO:1, a CDR_{H2} sequence of SEQ ID NO:6, and a CDR_{H3} sequence of SEQ ID NO: 13; and a CDR_{L1} sequence of SEQ ID NO: 16, a CDR_{L2} sequence of SEQ ID NO: 18, and a CDR_{L3} sequence of SEQ ID NO:21;
(vi) an antibody comprising a CDR_{H1} sequence of SEQ ID NO:1, a CDR_{H2} sequence of SEQ ID NO:8, and a CDR_{H3} sequence of SEQ ID NO: 13; and a CDR_{L1} sequence of SEQ ID NO: 16, a CDR_{L2} sequence of SEQ ID NO: 18, and a CDR_{L3} sequence of SEQ ID NO:21;
(vii) an antibody comprising a CDR_{H1} sequence of SEQ ID NO:1, a CDRH₂ sequence of SEQ ID NO:9, and a CDR_{H3} sequence of SEQ ID NO:13; and a CDR_{L1} sequence of SEQ ID NO:16, a CDR_{L2} sequence of SEQ ID NO: 18, and a CDR_{L3} sequence of SEQ ID NO:21;
(viii) an antibody comprising the heavy chain sequence of SEQ ID NO:47 or 49 or a variable region thereof, and the light chain sequence of SEQ ID NO:30 or the variable region thereof;
(ix) an antibody comprising the heavy chain sequence of SEQ ID NO:41, 42, 43, 44, 45, 46, 48, or 49 or a variable region thereof, and the light chain sequence of SEQ ID NO:29 or the variable region thereof;
(x) an antibody comprising the heavy chain sequence of SEQ ID NO:41, 42, 43, 44, or 45 or a variable region thereof, and the light chain sequence of SEQ ID NO:28 or the variable region thereof;
(xi) an antibody comprising the heavy chain sequence of SEQ ID NO:39, 40, 41, 42, 43, 44, or 45 or a variable region thereof, and the light chain sequence of SEQ ID NO:27 or the variable region thereof;
(xii) an antibody comprising the heavy chain sequence of SEQ ID NO:38 or the variable region thereof and the light chain sequence of SEQ ID NO:26 or the variable region thereof;
(xiii) an antibody comprising the heavy chain sequence of SEQ ID NO:37 or the variable region thereof, and the light chain sequence of SEQ ID NO:25 or the variable region thereof;
(xiv) an antibody comprising the heavy chain sequence of SEQ ID NO: 48, or the variable region thereof and the light chain sequence of SEQ ID NO: 29, or the variable region thereof; and
(xv) an antibody comprising the heavy chain sequence of SEQ ID NO: 47, or the variable region thereof and the light chain sequence of SEQ ID NO: 30, or the variable region thereof.

11. The method of claim 8, wherein the anti-GDF15 antibody is an antibody comprising a CDR_{H1} sequence of SEQ ID NO:1, a CDR_{H2} sequence of SEQ ID NO:7, and a CDR_{H3} sequence of SEQ ID NO: 13; and a CDR_{L1} sequence of SEQ ID NO:16, a CDR_{L2} sequence of SEQ ID NO: 18, and a CDR_{L3} sequence of SEQ ID NO:22.

12. The method of claim 8, wherein the anti-GDF15 antibody is an antibody comprising a CDR_{H1} sequence of SEQ ID NO: 1, a CDR_{H2} sequence of SEQ ID NO:8, and a CDR_{H3} sequence of SEQ ID NO: 13; and a CDR_{L1} sequence of SEQ ID NO:16, a CDR_{L2} sequence of SEQ ID NO: 18, and a CDR_{L3} sequence of SEQ ID NO:21.

13. The method of claim 8, wherein the anti-GDF15 antibody is an antibody comprising the heavy chain sequence of SEQ ID NO: 48 or the variable region thereof, and the light chain sequence of SEQ ID NO: 29, or the variable region thereof.

14. The method of claim 8, wherein the anti-GDF15 antibody is an antibody comprising the heavy chain sequence of SEQ ID NO: 47 or the variable region thereof and the light chain sequence of SEQ ID NO: 30, or the variable region thereof.

15. A GDF15 modulator, for use in reducing ocular tissue fibrosis in a subject in need thereof.

16. (canceled)

17. (canceled)

18. (canceled)

19. (canceled)

20. (canceled)

21. (canceled)

22. The GDF15 modulator of claim 15, wherein GDF15 modulator decreases or inhibits GDF15 activity.

23. The GDF15 modulator of claim 15, wherein the GDF15 modulator is an anti-GDF15 antibody.

24. (canceled)

25. The GDF15 modulator of claim 23, wherein the anti-GDF15 antibody is selected from:
(i) an antibody comprising a CDR_{H1} sequence of SEQ ID NO:1, a CDR_{H2} sequence of SEQ ID NO:7, and a CDR_{H3} sequence of SEQ ID NO: 13; and a CDR_{L1} sequence of SEQ ID NO: 16, a CDR_{L2} sequence of SEQ ID NO: 18, and a CDR_{L3} sequence of SEQ ID NO:22;
(ii) an antibody comprising a CDR_{H1} sequence of SEQ ID NO: 1, a CDR_{H2} sequence of SEQ ID NO:9, and a CDR_{H3} sequence of SEQ ID NO: 13; and a CDR_{L1} sequence of SEQ ID NO: 16, a sequence of SEQ ID NO:18, and a CDR_{L3} sequence of SEQ ID NO:22;
(iii) an antibody comprising a CDR_{H1} sequence of SEQ ID NO:1, a CDR_{H2} sequence of SEQ ID NO:4, and a CDR_{H3} sequence of SEQ ID NO:13; and a CDR_{L1} sequence of SEQ ID NO:16, a CDR_{L2} sequence of SEQ ID NO: 18, and a CDR_{L3} sequence of SEQ ID NO:21;
(iv) an antibody comprising a CDR_{H1} sequence of SEQ ID NO:1, a CDR_{H2} sequence of SEQ ID NO:5, and a CDR_{H3} sequence of SEQ ID NO:13; and a CDR_{L1} sequence of SEQ ID NO:16, a CDR_{L2} sequence of SEQ ID NO: 18, and a CDR_{L3} sequence of SEQ ID NO:21;
(v) an antibody comprising a CDR_{H1} sequence of SEQ ID NO: 1, a CDR_{H2} sequence of SEQ ID NO:6, and a CDR_{H3} sequence of SEQ ID NO: 13; and a CDR_{L1} sequence of SEQ ID NO: 16, a CDR_{L2} sequence of SEQ ID NO:18, and a CDR_{L3} sequence of SEQ ID NO:21;
(vi) an antibody comprising a CDR_{H1} sequence of SEQ ID NO: 1, a CDR_{H2} sequence of SEQ ID NO:8, and a CDR_{H3} sequence of SEQ ID NO: 13; and a CDR_{L1} sequence of SEQ ID NO: 16, a CDR_{L2} sequence of SEQ ID NO:18, and a CDR_{L3} sequence of SEQ ID NO:21;
(vii) an antibody comprising a CDR_{H1} sequence of SEQ ID NO:1, a CDR_{H2} sequence of SEQ ID NO:9, and a CDR_{H3} sequence of SEQ ID NO: 13; and a CDR_{L1} sequence of SEQ ID NO: 16, a CDR_{L2} sequence of SEQ ID NO:18, and a CDR_{L3} sequence of SEQ ID NO:21;
(viii) an antibody comprising the heavy chain sequence of SEQ ID NO:47 or 49 or a variable region thereof, and the light chain sequence of SEQ ID NO:30 or the variable region thereof;
(ix) an antibody comprising the heavy chain sequence of SEQ ID NO:41, 42, 43, 44, 45, 46, 48, or 49 or a variable region thereof, and the light chain sequence of SEQ ID NO:29 or the variable region thereof;
(x) an antibody comprising the heavy chain sequence of SEQ ID NO:41, 42, 43, 44, or 45 or a variable region thereof, and the light chain sequence of SEQ ID NO:28 or the variable region thereof;
(xi) an antibody comprising the heavy chain sequence of SEQ ID NO:39, 40, 41, 42, 43, 44, or 45 or a variable region thereof, and the light chain sequence of SEQ ID NO:27 or the variable region thereof;
(xii) an antibody comprising the heavy chain sequence of SEQ ID NO:38 or the variable region thereof, and the light chain sequence of SEQ ID NO:26 or the variable region thereof;
(xiii) an antibody comprising the heavy chain sequence of SEQ ID NO:37 or the variable region thereof, and the light chain sequence of SEQ ID NO:25 or the variable region thereof;
(xiv) an antibody comprising the heavy chain sequence of SEQ ID NO: 48 or the variable region thereof, and the light chain sequence of SEQ ID NO: 29 or the variable region thereof; and
(xv) an antibody comprising the heavy chain sequence of SEQ ID NO: 47 or the variable region thereof, and the light chain sequence of SEQ ID NO: 30 or the variable region thereof.

26. (canceled)

27. (canceled)

28. (canceled)

29. (canceled)

30. An ocular tissue fibrosis inhibitor, comprising a substance inhibiting action of GDF15 (growth differentiation factor 15) as an active ingredient.

31. The ocular tissue fibrosis inhibitor according to claim 30, wherein the substance inhibiting the action of the GDF15 is an anti-GDF15 antibody or an antagonist of a GDF15-specific receptor.

32. (canceled)


SEQUENCE LISTING (160 sequences)

SEQ ID NO: 1 - protein, 5 aa, Artificial Sequence
  Description of Artificial Sequence: Synthetic peptide
DYNMD

SEQ ID NO: 2 - protein, 7 aa, Artificial Sequence
  Description of Artificial Sequence: Synthetic peptide
TYGMGVS

SEQ ID NO: 3 - protein, 7 aa, Artificial Sequence
  Description of Artificial Sequence: Synthetic peptide
TYGMGVG

SEQ ID NO: 4 - protein, 17 aa, Artificial Sequence
  Description of Artificial Sequence: Synthetic peptide
QINPNNGGIFFNQKFKG

SEQ ID NO: 5 - protein, 17 aa, Artificial Sequence
  Description of Artificial Sequence: Synthetic peptide
QINPNNGGIFFNQKFQG

SEQ ID NO: 6 - protein, 17 aa, Artificial Sequence
  Description of Artificial Sequence: Synthetic peptide
QINPYNHLIFFNQKFQG

SEQ ID NO: 7 - protein, 17 aa, Artificial Sequence
  Description of Artificial Sequence: Synthetic peptide
QINPNNGLIFFNQKFQG

SEQ ID NO: 8 - protein, 17 aa, Artificial Sequence
  Description of Artificial Sequence: Synthetic peptide
QINPNNGLIFFNQKFKG

SEQ ID NO: 9 - protein, 17 aa, Artificial Sequence
  Description of Artificial Sequence: Synthetic peptide
QINPYNHLIFFNQKFKG

SEQ ID NO: 10 - protein, 16 aa, Artificial Sequence
  Description of Artificial Sequence: Synthetic peptide
HIYWDDDKRYNPSLKS

SEQ ID NO: 11 - protein, 16 aa, Artificial Sequence
  Description of Artificial Sequence: Synthetic peptide
HIYWDDDKRYNPSLKT

SEQ ID NO: 12 - protein, 16 aa, Artificial Sequence
  Description of Artificial Sequence: Synthetic peptide
DIWWDDDKYYNPSLKS

SEQ ID NO: 13 - protein, 11 aa, Artificial Sequence
  Description of Artificial Sequence: Synthetic peptide
EAITTVGAMDY

SEQ ID NO: 14 - protein, 9 aa, Artificial Sequence
  Description of Artificial Sequence: Synthetic peptide
RGYDDYWGY

SEQ ID NO: 15 - protein, 9 aa, Artificial Sequence
  Description of Artificial Sequence: Synthetic peptide
RGHYSAMDY

SEQ ID NO: 16 - protein, 11 aa, Artificial Sequence
  Description of Artificial Sequence: Synthetic peptide
RTSENLHNYLA

SEQ ID NO: 17 - protein, 11 aa, Artificial Sequence
  Description of Artificial Sequence: Synthetic peptide
KASQNVGTNVA

SEQ ID NO: 18 - protein, 7 aa, Artificial Sequence
  Description of Artificial Sequence: Synthetic peptide
DAKTLAD

SEQ ID NO: 19 - protein, 7 aa, Artificial Sequence
  Description of Artificial Sequence: Synthetic peptide
SASYRYS

SEQ ID NO: 20 - protein, 7 aa, Artificial Sequence
  Description of Artificial Sequence: Synthetic peptide
SPSYRYS

SEQ ID NO: 21 - protein, 9 aa, Artificial Sequence
  Description of Artificial Sequence: Synthetic peptide
QHFWSSPYT

SEQ ID NO: 22 - protein, 9 aa, Artificial Sequence
  Description of Artificial Sequence: Synthetic peptide
QHFWSDPYT

SEQ ID NO: 23 - protein, 9 aa, Artificial Sequence
  Description of Artificial Sequence: Synthetic peptide
QQYNNYPLT

SEQ ID NO: 24 - protein, 9 aa, Artificial Sequence
  Description of Artificial Sequence: Synthetic peptide
QQYNSYPHT

SEQ ID NO: 25 - protein, 214 aa, Artificial Sequence
  Description of Artificial Sequence: Synthetic polypeptide
DIQMTQSPASLSASVGETVTITCRTSENLHNYLAWYQQKQGKSPQLLVYDAKTLADGVPSRFSGSGSGTQYSLKINSLQPEDFGSYYCQHFWSSPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 26 - protein, 214 aa, Artificial Sequence
  Description of Artificial Sequence: Synthetic polypeptide
DIQMTQSPASLSASVGETVTITCRTSENLHNYLAWYQQKQGKSPQLLVYDAKTLADGVPSRFSGSGSGTQYSLKINSLQPEDFGSYYCQHFWSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 214 aa, Artificial Sequence
  Description of Artificial Sequence: Synthetic polypeptide
DIQMTQSPSSLSASVGDRVTITCRTSENLHNYLAWYQQKPGKSPKLLVYDAKTLADGVPSRFSGSGSGTDYTLTISSLQPEDFATYYCQHFWSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 214 aa, Artificial Sequence
  Description of Artificial Sequence: Synthetic polypeptide
DIQMTQSPSSLSASVGDRVTITCRTSENLHNYLAWYQQKPGKSPKLLIYDAKTLADGVPSRFSGSGSGTDYTLTISSLQPEDFATYYCQHFWSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 214 aa, Artificial Sequence
  Description of Artificial Sequence: Synthetic polypeptide
DIQMTQSPSSLSASVGDRVTITCRTSENLHNYLAWYQQKPGKAPKLLIYDAKTLADGVPSRFSGSGSGTDYTLTISSLQPEDFATYYCQHFWSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, Artificial Sequence
  Description of Artificial Sequence: Synthetic polypeptide
DIQMTQSPSSLSASVGDRVTITCRTSENLHNYLAWYQQKPGKSPKLLIYDAKTLADGVPSRFSGSGSGTDYTLTISSLQPEDFATYYCQHFWSDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, Artificial Sequence
  Description of Artificial Sequence: Synthetic polypeptide
DIVMTQSQKFMSTSVGDRVSVTCKASQNVGTNVAWFQQKPGQSPKALIYSASYRYSGVPDRFTGSGSGTDFILTISNVQSEDLAEYFCQQYNNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 214 aa, Artificial Sequence
  Description of Artificial Sequence: Synthetic polypeptide
DIVMTQSQKFMSTSVGDRVSVTCKASQNVGTNVAWFQQKPGQSPKALIYSASYRYSGVPDRFTGSGSGTDFILTISNVQSEDLAEYFCQQYNNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 214 aa, Artificial Sequence
  Description of Artificial Sequence: Synthetic polypeptide
DIQMTQSPSSLSASVGDRVTITCKASQNVGTNVAWFQQKPGKAPKSLIYSASYRYSGVPSRFSGSGSGTDFTLTISSLQPEDFATYYCQQYNNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 214 aa, Artificial Sequence
  Description of Artificial Sequence: Synthetic polypeptide
DIVMTQSQKFMSTSVGDRVSVTCKASQNVGTNVAWYQQKPGQSPKALIYSPSYRYSGVPDRFTGSGSGTDFTLTISNVQSEDLAEYFCQQYNSYPHTFGGGTKLEMKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 35 - protein, 214 aa, Artificial Sequence
  Description of Artificial Sequence: Synthetic polypeptide
DIVMTQSQKFMSTSVGDRVSVTCKASQNVGTNVAWYQQKPGQSPKALIYSPSYRYSGVPDRFTGSGSGTDFTLTISNVQSEDLAEYFCQQYNSYPHTFGGGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 214 aa, Artificial Sequence
  Description of Artificial Sequence: Synthetic polypeptide
DIQMTQSPSSLSASVGDRVTITCKASQNVGTNVAWFQQKPGKSPKALIYSPSYRYSGVPSRFSGSGSGTDFTLTISSLQPEDFATYFCQQYNSYPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 444 aa, Artificial Sequence
  Description of Artificial Sequence: Synthetic polypeptide
EVLLQQSGPELVKPGASVKIPCKASGYTFTDYNMDWVKQSHGKSLEWIGQINPNNGGIFFNQKFKGKATLTVDKSSNTAFMEVRSLTSEDTAVYYCAREAITTVGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 38 - protein, 450 aa, Artificial Sequence
  Description of Artificial Sequence: Synthetic polypeptide
EVLLQQSGPELVKPGASVKIPCKASGYTFTDYNMDWVKQSHGKSLEWIGQINPNNGGIFFNQKFKGKATLTVDKSSNTAFMEVRSLTSEDTAVYYCAREAITTVGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 450 aa, Artificial Sequence
  Description of Artificial Sequence: Synthetic polypeptide
QVQLVQSGAEVKKPGASVKVSCKASGYTFTDYNMDWVRQAPGKSLEWIGQINPNNGGIFFNQKFKGRATLTVDTSTNTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 450 aa, Artificial Sequence
  Description of Artificial Sequence: Synthetic polypeptide
QVQLVQSGAEVKKPGSSVKVSCKASGYTFTDYNMDWVRQAPGKSLEWIGQINPNNGGIFFNQKFKGRATLTVDKSTNTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 450 aa, Artificial Sequence
  Description of Artificial Sequence: Synthetic polypeptide
QVQLVQSGAEVKKPGASVKVSCKASGYTFTDYNMDWVRQAPGQGLEWMGQINPNNGGIFFNQKFKGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 450 aa, Artificial Sequence
  Description of Artificial Sequence: Synthetic polypeptide
QVQLVQSGAEVKKPGASVKVSCKASGYTFTDYNMDWVRQAPGQGLEWMGQINPNNGGIFFNQKFQGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 450 aa, Artificial Sequence
  Description of Artificial Sequence: Synthetic polypeptide
QVQLVQSGAEVKKPGASVKVSCKASGYTFTDYNMDWVRQAPGQSLEWMGQINPNNGGIFFNQKFQGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 450 aa, Artificial Sequence
  Description of Artificial Sequence: Synthetic polypeptide
QVQLVQSGAEVKKPGSSVKVSCKASGYTFSDYNMDWVRQAPGQGLEWMGQINPNNGGIFFNQKFKGRVTLTADKSTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 450 aa, Artificial Sequence
  Description of Artificial Sequence: Synthetic polypeptide
QVQLVQSGAEVKKPGSSVKVSCKASGYTFSDYNMDWVRQAPGQGLEWMGQINPNNGGIFFNQKFQGRVTLTADKSTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 450 aa, Artificial Sequence
  Description of Artificial Sequence: Synthetic polypeptide
QVQLVQSGAEVKKPGASVKVSCKASGYTFTDYNMDWVRQAPGQSLEWMGQINPYNHLIFFNQKFQGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 450 aa, Artificial Sequence
  Description of Artificial Sequence: Synthetic polypeptide
QVQLVQSGAEVKKPGASVKVSCKASGYTFTDYNMDWVRQAPGQSLEWMGQINPNNGLIFFNQKFQGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 450 aa, Artificial Sequence
  Description of Artificial Sequence: Synthetic polypeptide
QVQLVQSGAEVKKPGSSVKVSCKASGYTFSDYNMDWVRQAPGQGLEWMGQINPNNGLIFFNQKFKGRVTLTADKSTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 450 aa, Artificial Sequence
  Description of Artificial Sequence: Synthetic polypeptide
QVQLVQSGAEVKKPGSSVKVSCKASGYTFSDYNMDWVRQAPGQGLEWMGQINPYNHLIFFNQKFKGRVTLTADKSTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 443 aa, Artificial Sequence
  Description of Artificial Sequence: Synthetic polypeptide
QVTLKESGPGILQPSQTLSLTCSFSGFSLNTYGMGVSWIRQPSGKGLEWLAHIYWDDDKRYNPSLKSRLTISKDASNNRVFLKITSVDTADTATYYCAQRGYDDYWGYWGQGTLVTISAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 51 - protein, 449 aa, Artificial Sequence
  Description of Artificial Sequence: Synthetic polypeptide
QVTLKESGPALVKPTQTLTLTCTFSGFSLNTYGMGVSWIRQPPGKALEWLAHIYWDDDKRYNPSLKTRLTISKDTSKNQVVLTITNVDPVDTAVYYCAQRGYDDYWGYWGQGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 449 aa, Artificial Sequence
  Description of Artificial Sequence: Synthetic polypeptide
QVTLKESGPTLVKPTQTLTLTCTFSGFSLNTYGMGVSWIRQPPGKGLEWLAHIYWDDDKRYNPSLKSRLTITKDTSKNQVVLTITNMDPVDTATYYCAQRGYDDYWGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 443 aa, Artificial Sequence
  Description of Artificial Sequence: Synthetic polypeptide
QVTLKESGPGILQPSQTLSLTCSFSGFSLSTYGMGVGWIRQPSGKGLEWLADIWWDDDKYYNPSLKSRLTISKDTSSNEVFLKIAIVDTADTATYYCARRGHYSAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 54 - protein, 449 aa, Artificial Sequence
  Description of Artificial Sequence: Synthetic polypeptide
QVTLKESGPGILQPSQTLSLTCSFSGFSLSTYGMGVGWIRQPSGKGLEWLADIWWDDDKYYNPSLKSRLTISKDTSSNEVFLKIAIVDTADTATYYCARRGHYSAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 449 aa, Artificial Sequence
  Description of Artificial Sequence: Synthetic polypeptide
QVTLKESGPGILQPSQTLSLTCSFSGFSLNTYGMGVSWIRQPSGKGLEWLAHIYWDDDKRYNPSLKSRLTISKDASNNRVFLKITSVDTADTATYYCAQRGYDDYWGYWGQGTLVTISAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 447 aa, Artificial Sequence
  Description of Artificial Sequence: Synthetic polypeptide
QITLKESGPTLVKPTQTLTLTCTFSGFSLSTYGMGVGWIRQPPGKALEWLADIWWDDDKYYNPSLKSRLTITKDTSKNQVVLTMTNMDPVDTATYYCARRGHYSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQNVFSCSVMHEALHNHYTQKSLSLSPGK

SEQ ID NO: 57 - protein, 449 aa, Artificial Sequence
  Description of Artificial Sequence: Synthetic polypeptide
QVTLKESGPALVKPTQTLTLTCTFSGFSLSTYGMGVGWIRQPPGKALEWLADIWWDDDKYYNPSLKSRLTISKDTSKNQVVLTMTNMDPVDTAVYYCARRGHYSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 10 aa, Artificial Sequence
  Description of Artificial Sequence: Synthetic peptide
GYTFSSYNID

SEQ ID NO: 59 - protein, 17 aa, Artificial Sequence
  Description of Artificial Sequence: Synthetic peptide
GINPIFGTAFYNQKFQG

SEQ ID NO: 60 - protein, 11 aa, Artificial Sequence
  Description of Artificial Sequence: Synthetic peptide
EAITTVGAMDH

SEQ ID NO: 61 - protein, 11 aa, Artificial Sequence
  Description of Artificial Sequence: Synthetic peptide
RTSQSVHNYLA

SEQ ID NO: 62 - protein, 7 aa, Artificial Sequence
  Description of Artificial Sequence: Synthetic peptide
DASTRAD

SEQ ID NO: 63 - protein, 9 aa, Artificial Sequence
  Description of Artificial Sequence: Synthetic peptide
QQFWSWPWT

SEQ ID NO: 64 - protein, 120 aa, Artificial Sequence
  Description of Artificial Sequence: Synthetic polypeptide
QVQLVQSGAEVKKPGSSVKVSCKASGYTFSSYNISWVRQAPGQGLEWMGGINPINGLAFYNQKFQGRVTITADESTSTAYMELSSLRSEDTAVYYCAREAITTVGAMDYWGQGTLVTVSS

SEQ ID NO: 65 - protein, 120 aa, Artificial Sequence
  Description of Artificial Sequence: Synthetic polypeptide
QVQLVQSGAEVKKPGSSVKVSCKASGYTFSSYNIDWVRQAPGQGLEWMGQINPNNGLAFYNQKFQGRVTITADESTSTAYMELSSLRSEDTAVYYCAREAITTVGAMDYWGQGTLVTVSS

SEQ ID NO: 66 - protein, 120 aa, Artificial Sequence
  Description of Artificial Sequence: Synthetic polypeptide
QVQLVQSGAEVKKPGSSVKVSCKASGYTFSDYNIDWVRQAPGQGLEWMGQINPNNGLIFFNQKFQGRVTITADESTSTAYMELSSLRSEDTAVYYCAREVITTVGAMDYWGQGTLVTVSS

SEQ ID NO: 67 - protein, 120 aa, Artificial Sequence
  Description of Artificial Sequence: Synthetic polypeptide
QVQLVQSGAEVKKPGSSVKVSCKASGYTFSSYNIDWVRQAPGQGLEWMGGINPINGLIFFNQKFQGRVTITADESTSTAYMELSSLRSEDTAVYYCAREAITTVGAMDHWGQGTLVTVSS

SEQ ID NO: 68 - protein, 120 aa, Artificial Sequence
  Description of Artificial Sequence: Synthetic polypeptide
QVQLVQSGAEVKKPGSSVKVSCKASGYTFSDYNMDWVRQAPGQGLEWMGQINPNNGLANYNQKFQGRVTITADESTSTAYMELSSLRSEDTAVYYCAREAITTVGAMDYWGQGTLVTVSS

SEQ ID NO: 69 - protein, 120 aa, Artificial Sequence
  Description of Artificial Sequence: Synthetic polypeptide
QVQLVQSGAEVKKPGSSVKVSCKASGYTFTDYNIDWVRQAPGQGLEWMGQINPNNGLIFYNQKFQGRVTITADESTSTAYMELSSLRSEDTAVYYCAREAITTVGAMDYWGQGTLVTVSS

SEQ ID NO: 70 - protein, 120 aa, Artificial Sequence
  Description of Artificial Sequence: Synthetic polypeptide
QVQLVQSGAEVKKPGSSVKVSCKASGYTFTDYNIDWVRQAPGQGLEWMGQINPNNGLAFYNQKFQGRVTITADESTSTAYMELSSLRSEDNAVYYCAREAITTVGAMDYWGQGTLVTVSS

SEQ ID NO: 71 - protein, 120 aa, Artificial Sequence
  Description of Artificial Sequence: Synthetic polypeptide
QVQLVQSGAEVKKPGSSVKVSCKASGYTFSDYNMDWVRQAPGQGLEWMGQINPNNGLAFYNQKFQGRVTITADESTSTAYMELSSLRSEDTAVYYCAREAITTVGATDYWGQGTLVTVSS

SEQ ID NO: 72 - protein, 120 aa, Artificial Sequence
  Description of Artificial Sequence: Synthetic polypeptide
QVQLVQSGAEVKKPGSSVKVSCKASGYTFSSYNIDWVRQAPGQGLEWMGQINPINGLAFYNQKFQGRVTITADESTSTAYMELSSLRSEDTAVYYCAREAITTVGAMDYWGQGTLVTVSS

SEQ ID NO: 73 - protein, 120 aa, Artificial Sequence
  Description of Artificial Sequence: Synthetic polypeptide
QVQLVQSGAEVKKPGSSVKVSCKASGYTFSDYNMDWVRQAPGQGLEWMGGINPNNGLAFYNQKFQGRVTITADESTSTAYMELSSLRSEDTAVYYCAREAITTVGAMDYWGQGTLVTVSS

SEQ ID NO: 74 - protein, 120 aa, Artificial Sequence
  Description of Artificial Sequence: Synthetic polypeptide
QVQLVQSGAEVKKPGSSVKVSCKASGYTFSDYNMDWVRQAPGQGLEWMGQINPIFGLAFYAQKFQGRVTITADESTSTAYMELSSLRSEDTAVYYCAREVITTVGAMDYWGQGTLVTVSS

SEQ ID NO: 75 - protein, 120 aa, Artificial Sequence
  Description of Artificial Sequence: Synthetic polypeptide
QVQLVQSGAEVKKPGSSVKVSCKASGYTFSDYNIDWVRQAPGQGLEWMGGINPNNGLAFFNQKFQGRVTITADESTSTAYMELSSLRSEDTAVYYCAREAITTVGAMDYWGQGTLVTVSS

SEQ ID NO: 76 - protein, 120 aa, Artificial Sequence
  Description of Artificial Sequence: Synthetic polypeptide
QVQLVQSGAEVKKPGSSVKVSCKASGYTFSSYNISWVRQAPGQGLEWMGQINPNNGLAFYNQKFQGRVTITADESTSTAYMELSSLRSEDTAVYYCAREAITTVGAMEYWGQGTLVTVSS

SEQ ID NO: 77 - protein, 120 aa, Artificial Sequence
  Description of Artificial Sequence: Synthetic polypeptide
QVQLVQSGAEVKKPGSSVKVSCKASGYTFTSYNISWVRQAPGQGLEWMGQINPNNGLIFFAQKFQGRVTITADESTSTAYMELSSLRSEDTAVYYCAREAITTVGAMDQWGQGTLVTVSS

SEQ ID NO: 78 - protein, 120 aa, Artificial Sequence
  Description of Artificial Sequence: Synthetic polypeptide
QVQLVQSGAEVKKPGSSVKVSCKASGYTFSDYNISWVRQAPGQGLEWMGGINPIFGLAFYNQKFQGRVTITADESTSTAYMELSSLRSEDTAVYYCAREAITTVGAMDYWGQGTLVTVSS

SEQ ID NO: 79 - protein, 120 aa, Artificial Sequence
  Description of Artificial Sequence: Synthetic polypeptide
QVQLVQSGAEVKKPGSSVKVSCKASGYTFTDYNISWVRQAPGQGLEWMGQINPNNGLAFYAQKFQGRVTITADESTSTAYMELSSLRSEDTAVYYCAREFITTVGAMDYWGQGTLVTVSS

SEQ ID NO: 80 - protein, 120 aa, Artificial Sequence
  Description of Artificial Sequence: Synthetic polypeptide
QVQLVQSGAEVKKPGSSVKVSCKASGYTFSDYNMDWVRQAPGQGLEWMGGINPNNGTAFYAQKFQGRVTITADESTSTAYMELSSLRSEDTAVYYCAREAITTVGAMDQWGQGTLVTVSS

SEQ ID NO: 81 - protein, 120 aa, Artificial Sequence
  Description of Artificial Sequence: Synthetic polypeptide
QVQLVQSGAEVKKPGSSVKVSCKASGYTFSSYNIDWVRQAPGQGLEWMGQINPNNGLANYAQKFQGRVTITADESTSTAYMELSSLRSEDTAVYYCAREAITTIGAMDYWGQGTLVTVSS

SEQ ID NO: 82 - protein, 120 aa, Artificial Sequence
  Description of Artificial Sequence: Synthetic polypeptide
QVQLVQSGAEVKKPGSSVKVSCKASGYTFTSYNIDWVRQAPGQGLEWMGQINPNNGLAFYNQKFQGRVTITADESTSTAYMELSSLRSEDTAVYYCAREQITTVGAMDYWGQGTLVTVSS

SEQ ID NO: 83 - protein, 120 aa, Artificial Sequence
  Description of Artificial Sequence: Synthetic polypeptide
QVQLVQSGAEVKKPGSSVKVSCKASGYTFSSYNIDWVRQAPGQGLEWMGGINPIFGLAFYNQKFQGRVTITADESTSTAYMELSSLRSEDTAVYYCAREAITTVGAMDPWGQGTLVTVSS

SEQ ID NO: 84 - protein, 120 aa, Artificial Sequence
  Description of Artificial Sequence: Synthetic polypeptide
QVQLVQSGAEVKKPGSSVKVSCKASGYTFSSYNIDWVRQAPGQGLEWMGGINPIFGTAFYNQKFQGRVTITADESTSTAYMELSSLRSEDTAVYYCAREAITTVGAMDHWGQGTLVTVSS

SEQ ID NO: 85 - protein, 107 aa, Artificial Sequence
  Description of Artificial Sequence: Synthetic polypeptide
EIVLTQSPATLSLSPGERATLSCRASQSVHSYLAWYQQKPGQAPRLLIYDASNRATGIPARFSGSGSGTDFTLTISSLEPEDFAVYYCQQFWSWPWTFGQGTKVEIK

SEQ ID NO: 86 - protein, 107 aa, Artificial Sequence
  Description of Artificial Sequence: Synthetic polypeptide
EIVLTQSPATLSLSPGERATLSCRTSQNVHSYLAWYQQKPGQAPRLLIYDASTRADGIPARFSGSGSGTDFTLTISSLEPEDFAVYYCQQFWSDPWTFGQGTKVEIK

SEQ ID NO: 87 - protein, 107 aa, Artificial Sequence
  Description of Artificial Sequence: Synthetic polypeptide
EIVLTQSPATLSLSPGERATLSCRTSQSVHSYLAWYQQKPGQAPRLLIYDAKTRADGIPARFSGSGSGTDFTLTISSLEPEDFAVYYCQQFSSDPYTFGQGTKVEIK

SEQ ID NO: 88 - protein, 107 aa, Artificial Sequence
  Description of Artificial Sequence: Synthetic polypeptide
EIVLTQSPATLSLSPGERATLSCRTSENVHSYLAWYQQKPGQAPRLLIYDASNLADGIPARFSGSGSGTDFTLTISSLEPEDFAVYYCQQFWSDPYTFGQGTKVEIK

SEQ ID NO: 89 - protein, 107 aa, Artificial Sequence
  Description of Artificial Sequence: Synthetic polypeptide
EIVLTQSPATLSLSPGERATLSCRASQNLHSYLAWYQQKPGQAPRLLIYDASTRADGIPARFSGSGSGTDFTLTISSLEPEDFAVYYCQQFWSDPYTFGQGTKVEIK

SEQ ID NO: 90 - protein, 107 aa, Artificial Sequence
  Description of Artificial Sequence: Synthetic polypeptide
EIVLTQSPATLSLSPGERATLSCRASQNVHSYLAWYQQKPGQAPRLLIYDASTRADGIPARFSGSGSGTDFTLTISSLEPEDFAVYYCQQFWNDPYTFGQGTKVEIK

SEQ ID NO: 91 - protein, 107 aa, Artificial Sequence
  Description of Artificial Sequence: Synthetic polypeptide
EIVLTQSPATLSLSPGERATLSCRTSQSVHSYLAWYQQKPGQAPRLLIYDAKTRATGIPARFSGSGSGTDFTLTISSLEPEDFAVYYCQQFSSDPYTFGQGTKVEIK

SEQ ID NO: 92 - protein, 107 aa, Artificial Sequence
  Description of Artificial Sequence: Synthetic polypeptide
EIVLTQSPATLSLSPGERATLSCRTSQNVHSYLAWYQQKPGQAPRLLIYDASNLADGIPARFSGSGSGTDFTLTISSLEPEDFAVYYCQQFSNDPWTFGQGTKVEIK

SEQ ID NO: 93 - protein, 107 aa, Artificial Sequence
  Description of Artificial Sequence: Synthetic polypeptide
EIVLTQSPATLSLSPGERATLSCRTSQNVHNYLAWYQQKPGQAPRLLIYDASNRATGIPARFSGSGSGTDFTLTISSLEPEDFAVYYCQQFWSDPYTFGQGTKVEIK

SEQ ID NO: 94 - protein, 107 aa, Artificial Sequence
  Description of Artificial Sequence: Synthetic polypeptide
EIVLTQSPATLSLSPGERATLSCRTSESVHSYLAWYQQKPGQAPRLLIYDASNRATGIPARFSGSGSGTDFTLTISSLEPEDFAVYYCQQFWNWPWTFGQGTKVEIK

SEQ ID NO: 95 - protein, 107 aa, Artificial Sequence
  Description of Artificial Sequence: Synthetic polypeptide
EIVLTQSPATLSLSPGERATLSCRTSQSVHNYLAWYQQKPGQAPRLLIYDASTRADGIPARFSGSGSGTDFTLTISSLEPEDFAVYYCQQFSSDPYTFGQGTKVEIK

SEQ ID NO: 96 - protein, 107 aa, Artificial Sequence
  Description of Artificial Sequence: Synthetic polypeptide
EIVLTQSPATLSLSPGERATLSCRTSQSLHSYLAWYQQKPGQAPRLLIYDASNRATGIPARFSGSGSGTDFTLTISSLEPEDFAVYYCQQFWNDPWTFGQGTKVEIK

SEQ ID NO: 97 - protein, 107 aa, Artificial Sequence
  Description of Artificial Sequence: Synthetic polypeptide
EIVLTQSPATLSLSPGERATLSCRTSQNVHSYLAWYQQKPGQAPRLLIYDAKNRADGIPARFSGSGSGTDFTLTISSLEPEDFAVYYCQQFWSDPYTFGQGTKVEIK

SEQ ID NO: 98 - protein, 107 aa, Artificial Sequence
  Description of Artificial Sequence: Synthetic polypeptide
EIVLTQSPATLSLSPGERATLSCRTSQNVHSYLAWYQQKPGQAPRLLIYDASNRADGIPARFSGSGSGTDFTLTISSLEPEDFAVYYCQQFWNDPYTFGQGTKVEIK

SEQ ID NO: 99 - protein, 107 aa, Artificial Sequence
  Description of Artificial Sequence: Synthetic polypeptide
EIVLTQSPATLSLSPGERATLSCRTSENVHSYLAWYQQKPGQAPRLLIYDASTLATGIPARFSGSGSGTDFTLTISSLEPEDFAVYYCQQFWSWPWTFGQGTKVEIK

SEQ ID NO: 100 - protein, 107 aa, Artificial Sequence
  Description of Artificial Sequence: Synthetic polypeptide
EIVLTQSPATLSLSPGERATLSCRTSQSVSNYLAWYQQKPGQAPRLLIYDAKNRATGIPARFSGSGSGTDFTLTISSLEPEDFAVYYCQQFWNDPWTFGQGTKVEIK

SEQ ID NO: 101 - protein, 107 aa, Artificial Sequence
  Description of Artificial Sequence: Synthetic polypeptide
EIVLTQSPATLSLSPGERATLSCRTSESVSSYLAWYQQKPGQAPRLLIYDAKTRADGIPARFSGSGSGTDFTLTISSLEPEDFAVYYCQQFWSWPWTFGQGTKVEIK

SEQ ID NO: 102 - protein, 107 aa, Artificial Sequence
  Description of Artificial Sequence: Synthetic polypeptide
EIVLTQSPATLSLSPGERATLSCRTSESVHSYLAWYQQKPGQAPRLLIYDASTRADGIPARFSGSGSGTDFTLTISSLEPEDFAVYYCQQFWSDPYTFGQGTKVEIK

SEQ ID NO: 103 - protein, 107 aa, Artificial Sequence
  Description of Artificial Sequence: Synthetic polypeptide
EIVLTQSPATLSLSPGERATLSCRASQSLSSYLAWYQQKPGQAPRLLIYDAKNRADGIPARFSGSGSGTDFTLTISSLEPEDFAVYYCQQFSSDPYTFGQGTKVEIK

SEQ ID NO: 104 - protein, 107 aa, Artificial Sequence
  Description of Artificial Sequence: Synthetic polypeptide
EIVLTQSPATLSLSPGERATLSCRASQNVHNYLAWYQQKPGQAPRLLIYDASNRADGIPARFSGSGSGTDFTLTISSLEPEDFAVYYCQQFWSWPWTFGQGTKVEIK

SEQ ID NO: 105 - protein, 107 aa, Artificial Sequence
  Description of Artificial Sequence: Synthetic polypeptide
EIVLTQSPATLSLSPGERATLSCRTSQSVHNYLAWYQQKPGQAPRLLIYDASTRADGIPARFSGSGSGTDFTLTISSLEPEDFAVYYCQQFWSWPWTFGQGTKVEIK

SEQ ID NO: 106 - protein, 449 aa, Artificial Sequence
  Description of Artificial Sequence: Synthetic polypeptide
QVQLVQSGAEVKKPGSSVKVSCKASGYTFSSYNIDWVRQAPGQGLEWMGGINPIFGTAFYNQKFQGRVTITADESTSTAYMELSSLRSEDTAVYYCAREAITTVGAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 214 aa, Artificial Sequence
  Description of Artificial Sequence: Synthetic polypeptide
EIVLTQSPATLSLSPGERATLSCRTSQSVHNYLAWYQQKPGQAPRLLIYDASTRADGIPARFSGSGSGTDFTLTISSLEPEDFAVYYCQQFWSWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 16 aa, Artificial Sequence
  Description of Artificial Sequence: Synthetic peptide
GNPIFGTAFYNQKFQG

SEQ ID NO: 109 - protein, 119 aa, Artificial Sequence
  Description of Artificial Sequence: Synthetic polypeptide
QVQLVQSGAEVKKPGSSVKVSCKASGYTFSSYNIDWVRQAPGQGLEWMGGNPIFGTAFYNQKFQGRVTITADESTSTAYMELSSLRSEDTAVYYCAREAITTVGAMDHWGQGTLVTVSS

SEQ ID NO: 110 - protein, 448 aa, Artificial Sequence
  Description of Artificial Sequence: Synthetic polypeptide
QVQLVQSGAEVKKPGSSVKVSCKASGYTFSSYNIDWVRQAPGQGLEWMGGNPIFGTAFYNQKFQGRVTITADESTSTAYMELSSLRSEDTAVYYCAREAITTVGAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 5 aa, Artificial Sequence
  Description of Artificial Sequence: Synthetic peptide
DYWIS

SEQ ID NO: 112 - protein, 17 aa, Artificial Sequence
  Description of Artificial Sequence: Synthetic peptide
IIDPSGSYTIYSPSFQG

SEQ ID NO: 113 - protein, 10 aa, Artificial Sequence
  Description of Artificial Sequence: Synthetic peptide
VSYYGGYFDI

SEQ ID NO: 114 - protein, 11 aa, Artificial Sequence
  Description of Artificial Sequence: Synthetic peptide
RASQSISNNLN

SEQ ID NO: 115 - protein, 7 aa, Artificial Sequence
  Description of Artificial Sequence: Synthetic peptide
AASNLQS

SEQ ID NO: 116 - protein, 9 aa, Artificial Sequence
  Description of Artificial Sequence: Synthetic peptide
FQLDHSPFT

SEQ ID NO: 117 - protein, 119 aa, Artificial Sequence
  Description of Artificial Sequence: Synthetic polypeptide
EVQLVQSGAEVKKPGESLKISCKGSGYSFTDYWISWVRQMPGKGLEWMGIIDPSGSYTIYSPSFQGQVTISADKSISTAYLQWSSLKASDTAMYYCARVSYYGGYFDIWGQGTLVTVSS

SEQ ID NO: 118 - protein, 126 aa, Artificial Sequence
  Description of Artificial Sequence: Synthetic polypeptide
QVQLVQSGAEVKKPGSSVKVSCKASGGTFSSHYINWVRQAPGQGLEWMGGIIPAFGGANYAQKFQGRVTITADESTSTAYMELSSLRSEDTAVYYCARFGSVYVSRYSSYYHMDVWGQGTLVTVSS

SEQ ID NO: 119 - protein, 123 aa, Artificial Sequence
  Description of Artificial Sequence: Synthetic polypeptide
QVQLVQSGAEVKKPGSSVKVSCKASGGTFRSYAVSWVRQAPGQGLEWMGGIIPIFGTANYAQKFQGRVTITADESTSTAYMELSSLRSEDTAVYYCARGPIIMGYQFGLFDHWGQGTLVTVSS

SEQ ID NO: 120 - protein, 123 aa, Artificial Sequence; identical to SEQ ID NO: 119
  Description of Artificial Sequence: Synthetic polypeptide

SEQ ID NO: 121 - protein, 119 aa, Artificial Sequence
  Description of Artificial Sequence: Synthetic polypeptide
EVQLVQSGAEVKKPGESLKISCKGSGYSFTSYWIGWVRQMPGKGLEWMGVIDPDGSYTIYSPSFQGQVTISADKSISTAYLQWSSLKASDTAMYYCARYGRYGTYFDYWGQGTLVTVSS

SEQ ID NO: 122 - protein, 119 aa, Artificial Sequence
  Description of Artificial Sequence: Synthetic polypeptide
EVQLVQSGAEVKKPGESLKISCKGSGYSFTSYWIGWVRQMPGKGLEWMGVIDPGGSYTIYSPSFQGQVTISADKSISTAYLQWSSLKASDTAMYYCARYGRYGTYFDYWGQGTLVTVSS

SEQ ID NO: 123 - protein, 119 aa, Artificial Sequence
  Description of Artificial Sequence: Synthetic polypeptide
EVQLVQSGAEVKKPGESLKISCKGSGYSFTSYWIGWVRQMPGKGLEWMGVIDPSGSYTIYSPSFQGQVTISADKSISTAYLQWSSLKASDTAMYYCARYGRYGTYFDYWGQGTLVTVSS

SEQ ID NO: 124 - protein, 107 aa, Artificial Sequence
  Description of Artificial Sequence: Synthetic polypeptide
DIQMTQSPSSLSASVGDRVTITCRASQSISNNLNWYQQKPGKAPKLLIYAASNLQSGVPSRFSGSGSGTDFTLTISSLQPEDFATYYCFQLDHSPFTFGQGTKVEIK

SEQ ID NO: 125 - protein, 107 aa, Artificial Sequence
  Description of Artificial Sequence: Synthetic polypeptide
DIQMTQSPSSLSASVGDRVTITCRASQTIYRSLAWYQQKPGKAPKLLIYGASILQSGVPSRFSGSGSGTDFTLTISSLQPEDFATYYCLQRYTSPFTFGQGTKVEIK

SEQ ID NO: 126 - protein, 107 aa, Artificial Sequence
  Description of Artificial Sequence: Synthetic polypeptide
SYELTQPLSVSVALGQTARITCSGDNIGSHIVSWYQQKPGQAPVLVIYDKSNRPSGIPERFSGSNSGNTATLTISRAQAGDEADYYCQTWDSIGSVVFGGGTKLTVL

SEQ ID NO: 127 - protein, 107 aa, Artificial Sequence
  Description of Artificial Sequence: Synthetic polypeptide
SYELTQPPSVSVSPGQTASITCSGDNIGSHIVSWYQQKPGQSPVLVIYDKSNRPSGIPERFSGSNSGNTATLTISGTQAMDEADYYCQTWDSIGSVVFGGGTKLTVL

SEQ ID NO: 128 - protein, 109 aa, Artificial Sequence
  Description of Artificial Sequence: Synthetic polypeptide
QSVLTQPPSVSGAPGQRVTISCSGSSSNIGVLYVNWYQQLPGTAPKLLIYSNDNRPSGVPDRFSGSKSGTSASLAITGLQAEDEADYYCQSWDSSSNYVFGGGTKLTVL

SEQ ID NO: 129 - protein, 109 aa, Artificial Sequence; identical to SEQ ID NO: 128
  Description of Artificial Sequence: Synthetic polypeptide

SEQ ID NO: 130 - protein, 109 aa, Artificial Sequence; identical to SEQ ID NO: 128
  Description of Artificial Sequence: Synthetic polypeptide

SEQ ID NO: 131 - protein, 449 aa, Artificial Sequence
  Description of Artificial Sequence: Synthetic polypeptide
EVQLVQSGAEVKKPGESLKISCKGSGYSFTDYWISWVRQMPGKGLEWMGIIDPSGSYTIYSPSFQGQVTISADKSISTAYLQWSSLKASDTAMYYCARVSYYGGYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 214 aa, Artificial Sequence
  Description of Artificial Sequence: Synthetic polypeptide
DIQMTQSPSSLSASVGDRVTITCRASQSISNNLNWYQQKPGKAPKLLIYAASNLQSGVPSRFSGSGSGTDFTLTISSLQPEDFATYYCFQLDHSPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 10 aa, Artificial Sequence
  Description of Artificial Sequence: Synthetic peptide
GYTFTDYGVI

SEQ ID NO: 134 - protein, 17 aa, Artificial Sequence
  Description of Artificial Sequence: Synthetic peptide
WINTYTGEPTYADDLKG

SEQ ID NO: 135 - protein, 9 aa, Artificial Sequence
  Description of Artificial Sequence: Synthetic peptide
RYGPEDIDY

SEQ ID NO: 136 - protein, 5 aa, Artificial Sequence
  Description of Artificial Sequence: Synthetic peptide
DYGVI

SEQ ID NO: 137 - protein, 7 aa, Artificial Sequence
  Description of Artificial Sequence: Synthetic peptide
GYTFTDY

SEQ ID NO: 138 - protein, 4 aa, Artificial Sequence
  Description of Artificial Sequence: Synthetic peptide
TYTG

SEQ ID NO: 139 - protein, 7 aa, Artificial Sequence
  Description of Artificial Sequence: Synthetic peptide
YGPEDID

SEQ ID NO: 140 - protein, 8 aa, Artificial Sequence
  Description of Artificial Sequence: Synthetic peptide
GYTFTDYG

SEQ ID NO: 141 - protein, 8 aa, Artificial Sequence
  Description of Artificial Sequence: Synthetic peptide
INTYTGEP

SEQ ID NO: 142 - protein, 11 aa, Artificial Sequence
  Description of Artificial Sequence: Synthetic peptide
ARRYGPEDIDY

SEQ ID NO: 143 - protein, 6 aa, Artificial Sequence
  Description of Artificial Sequence: Synthetic peptide
TDYGVI

SEQ ID NO: 144 - protein, 13 aa, Artificial Sequence
  Description of Artificial Sequence: Synthetic peptide
WMGWINTYTGEPT

SEQ ID NO: 145 - protein, 10 aa, Artificial Sequence
  Description of Artificial Sequence: Synthetic peptide
ARRYGPEDID

SEQ ID NO: 146 - protein, 10 aa, Artificial Sequence
  Description of Artificial Sequence: Synthetic peptide
WINTYTGEPT

SEQ ID NO: 147 - protein, 15 aa, Artificial Sequence
  Description of Artificial Sequence: Synthetic peptide
RASESVDNYGISFMS

SEQ ID NO: 148 - protein, 7 aa, Artificial Sequence
  Description of Artificial Sequence: Synthetic peptide
AASHQGS

SEQ ID NO: 149 - protein, 9 aa, Artificial Sequence
  Description of Artificial Sequence: Synthetic peptide
LQSKEVPWT

SEQ ID NO: 150 - protein, 11 aa, Artificial Sequence
  Description of Artificial Sequence: Synthetic peptide
SESVDNYGISF

SEQ ID NO: 151 - protein, 3 aa, Artificial Sequence
  Description of Artificial Sequence: Synthetic peptide
AAS

SEQ ID NO: 152 - protein, 6 aa, Artificial Sequence
  Description of Artificial Sequence: Synthetic peptide
SKEVPW

SEQ ID NO: 153 - protein, 10 aa, Artificial Sequence
  Description of Artificial Sequence: Synthetic peptide
ESVDNYGISF

SEQ ID NO: 154 - protein, 11 aa, Artificial Sequence
  Description of Artificial Sequence: Synthetic peptide
DNYGISFMSWF

SEQ ID NO: 155 - protein, 10 aa, Artificial Sequence
  Description of Artificial Sequence: Synthetic peptide
LLIYAASHQG

SEQ ID NO: 156 - protein, 8 aa, Artificial Sequence
  Description of Artificial Sequence: Synthetic peptide
LQSKEVPW

SEQ ID NO: 157 - protein, 118 aa, Artificial Sequence
  Description of Artificial Sequence: Synthetic polypeptide
QIQLVQSGPELKKPGETVKISCKASGYTFTDYGVIWVKQAPGKALKWMGWINTYTGEPTYADDLKGRFAFSLETSASSASLQINNLKNEDTATYFCARRYGPEDIDYWGQGTTLTVSS

SEQ ID NO: 158 - protein, 111 aa, Artificial Sequence
  Description of Artificial Sequence: Synthetic polypeptide
DIVLTQSPVSLAVSLGQRATISCRASESVDNYGISFMSWFQQKPGQPPKLLIYAASHQGSGVPARFSGSGSGTDFSLNIHPMEEDDSAMYFCLQSKEVPWTFGGGTKLEIK

SEQ ID NO: 159 - protein, 118 aa, Artificial Sequence
  Description of Artificial Sequence: Synthetic polypeptide
QVQLVQSGAEVKKPGSSVKVSCKASGYTFTDYGVIWVRQAPGQGLEWMGWINTYTGEPTYADDLKGRVTFTADESTSTAYMELSSLRSEDTAVYYCARRYGPEDIDYWGQGTTVTVSS

SEQ ID NO: 160 - protein, 111 aa, Artificial Sequence
  Description of Artificial Sequence: Synthetic polypeptide
DVVLTQSPLSLPVTLGQPASISCRASESVDNYGISFMSWFQQRPGQSPRLLIYAASHQGSGVPDRFSGSGSGTDFTLKISRVEAEDVGVYFCLQSKEVPWTFGGGTKVEIK